(19) United States
(12) Patent Application Publication
(10) Pub. No.: US 2023/0157977 A1
(43) Pub. Date: May 25, 2023

(54) REPEATED TOPICAL APPLICATION OF CAPSAICIN PATCH FOR TREATING INITIAL NON-RESPONDERS

(71) Applicant: GRUENENTHAL GMBH, Aachen (DE)

(72) Inventors: Marielle EERDEKENS, Dilsen-Stokkem (BE); Sylvia ENGELEN, NM Vijlen (NL)

(73) Assignee: GRUENENTHAL GMBH, Aachen (DE)

(21) Appl. No.: 17/912,280

(22) PCT Filed: Mar. 17, 2021
(86) PCT No.: PCT/EP2021/056776

§ 371 Date: Sep. 16, 2022

(30) Foreign Application Priority Data
Mar. 17, 2020 (EP) 20163651.1
Apr. 3, 2020 (EP) 20167982.6
May 6, 2020 (EP) 20173133.8

(51) Int. Cl. A61K 31/165; A61P 25/02; A61K 9/70

(52) U.S. Cl. CPC A61K 31/165; A61P 25/02; A61K 9/7023; A61K 9/0014

(57) ABSTRACT

The invention relates to repeated treatment of a neuropathic condition, preferably peripheral neuropathic pain, by application of one or more high-concentration capsaicin and/or capsaicinoid topical dose units, preferably high-concentration capsaicin and/or capsaicinoid patches, to patients who previously did not respond or insufficiently responded to a previous application of one or more high-concentration capsaicin and/or capsaicinoid topical dose units, preferably high-concentration capsaicin and/or capsaicinoid patches or any other high-concentration capsaicin and/or capsaicinoid formulation previously topically administered.

### CROSS-REFERENCES

Priority is claimed of European patent application no. 20 163 651.1 that was filed on Mar. 17, 2020, European patent application no. 20 167 982.6 that was filed on Apr. 3, 2020, and European patent application no. 20 173 133.8 that was filed on May 6, 2020.

### FIELD OF THE INVENTION

The invention relates to repeated treatment of a neuropathic condition, preferably peripheral neuropathic pain, by application of one or more high-concentration capsaicin and/or capsaicinoid topical dose units (topical systems), preferably high-concentration capsaicin and/or capsaicinoid patches, to patients who previously did not respond or insufficiently responded to a previous application of one or more high-concentration capsaicin and/or capsaicinoid topical dose units, preferably high-concentration capsaicin and/or capsaicinoid patches or any other high-concentration capsaicin and/or capsaicinoid formulation previously topically administered.

### BACKGROUND ART

Capsaicin is a highly selective agonist for the transient receptor potential vanilloid 1 receptor (TRPV1). The initial effect of capsaicin is the activation of TRPV1-expressing cutaneous nociceptors, which results in pungency and erythema due to the release of vasoactive neuropeptides. Following capsaicin exposure, cutaneous nociceptors become less sensitive to a variety of stimuli. These later-stage effects of capsaicin are frequently referred to as “desensitization” and are thought to underlie the pain relief. Sensations from non TRPV1-expressing cutaneous nerves are expected to remain unaltered, including the ability to detect mechanical and vibratory stimuli. Capsaicin-induced alterations in cutaneous nociceptors are reversible and it has been reported and observed that normal function (the detection of noxious sensations) returns within weeks in healthy volunteers. F. Peppin et al., Ther Adv Neurol Disord (2014) 7(1) 22-32 review the use of capsaicinoids in the treatment of neuropathic pain.

Low-concentration capsaicin creams, lotions, and patches (capsaicin concentrations of 0.025 wt.-% to 0.1% wt.-%) intended for daily topical application have been available in most countries since the early 1980s. These topical formulations are usually self-administered medications and often without the requirement of a prescription. Clinical studies have revealed that three to five topical skin applications per day for periods of two to six weeks have modest beneficial effects against various pain syndromes, including post-herpetic neuralgia, diabetic neuropathy, and chronic musculoskeletal pain (see V. Fattori et al., Molecules 2016, 21, 844, 1-33).

Medium-concentration capsaicin patches have been tested in clinical trials. For example, J.-Y. Moon et al., Pain Physician 2017, 20:27-35 report about efficacy and safety of 0.625% (50 μg/cm²) and 1.25% (100 μg/cm²) capsaicin patch in peripheral neuropathic pain.

High-concentration capsaicin topical dosage forms such as liquids, creams or oils are known from e.g. WO 2004/091521, WO 2005/117981, WO 2013/036961, WO 2015/160941, U.S. Pat. Nos. 6,248,788, 7,771,760, 8,367,733, 8,802,736.

W. R. Robbins et al., Anesth Analg 1998 86 579-83 report about treatment of intractable pain with topical large-dose capsaicin. Webster et al., Journal of Pain, 3(4) S72 2012 and M. Wallace et al., PainWeek 2014 report about clinical trials with a topical liquid formulation of capsaicin in patients with postherpetic neuralgia.

High-concentration capsaicin topical cream (8%) has been developed and suggested for the treatment of myofascial pain syndrome. In a placebo-controlled study, patients were asked to point out the most painful trigger point during the physical examination. This trigger was demarcated by a 24 mm diameter circular mold with a stylus for use on the skin. Capsaicin 8% cream was applied in an amount of 10 g for 30 minutes on the demarcated skin area. The authors concluded that capsaicin 8%, as topically applied in patients with myofascial pain syndrome, showed significant analgesic effect. This analgesia persisted for almost two months after a single application without producing significant adverse effects (V. Romero et al., Rev Bras Anestesiol. 2019, 69(5), 432-438).

High-concentration capsaicin patches are known from e.g. US 2001 00002406, U.S. Pat. No. 6,248,788, US 2004 0202707 and US 2014 0335150.

High-concentration capsaicin patch (179 mg or 8 wt.-%) is commercially available (Qutenza®) with a capsaicin concentration about 100 times greater than conventional creams (see e.g. U.S. Pat. No. 6,239,180). Each 280 cm² cutaneous patch contains a total of 179 mg of capsaicin or 640 micrograms of capsaicin per cm² of patch. Each patch is 14 cm×20 cm and consists of an adhesive side containing the active substance and an outer surface backing layer. The adhesive side is covered with a removable, clear, unprinted, diagonally cut, release liner. The cutaneous patch is applied to the most painful skin areas (using up to a maximum of 4 patches).

High-concentration capsaicin patch is indicated for the treatment of peripheral neuropathic pain in the EU and for the treatment of post herpetic neuralgia in the US, in adults either alone or in combination with other medicinal products for their treatment of pain. The efficacy of a single application of high-concentration capsaicin patch has been shown to be maintained for up to 12 weeks in multiple randomized controlled clinical trials. High-concentration capsaicin patch treatment can be repeated approximately every 3 months.

The 8% capsaicin patch in the management of neuropathic pain has been reviewed by several authors. G. Baranidharan et al., Ther Adv Neurol Disord 2013 6(5) 287-297 review the high-concentration capsaicin patch and experience in its use in the management of neuropathic pain. T. Wagner et al., Pain Medicine 2013 14 1202-1211 provide a retrospective analysis a the capsaicin 8% patch for neuropathic pain in clinical practice. H. A. Blair, Drugs 2018 78 1489-1500 reviews capsaicin 8% dermal patch in the treatment of peripheral neuropathic pain.

A. Papagianni et al., PAIN Reports 2018 3 e644 1-9 report that capsaicin 8% patch reversibly reduces A-delta fiber evoked potential amplitudes. P. Anand et al. Journal of Pain Research 2019 12 2039-2052 relates to pain relief and disease modification in rational treatment of chemotherapy-induced peripheral neuropathy with capsaicin 8% patch.

The 8% capsaicin patch has been subject to various clinical trials. For example, M. Miroslav et al., Pain Medicine 2010 11 600-608 report about a randomized double-blind controlled study with open label extension regarding NGX-4010, a high-concentration capsaicin patch. L. R. Webster et al., Diab Res Clin Pract 2011 93 187-197 report about efficacy, safety and tolerability of NGX-4010 capsaicin 8% patch in an open-label study of patients with peripheral neuropathic pain. D. B. Clifford et al., J Acquir Immune Defic Syndr 2012 59(2) 126-133 report about a randomized double-blind controlled study of NGX-4010, a capsaicin 8% dermal patch for the treatment of painful HIV-associated distal sensory polyneuropathy. S. Brown et al., AIDS Res Ther. 2013 10(1):5 report about NGX-4010, a capsaicin 8% patch, for the treatment of painful HIV-associated distal sensory polyneuropathy: integrated analysis of two phase III, randomized, controlled trials.

Several clinical trials were focused on the treatment of postherpetic neuralgia. L. R. Webster et al., BMC Neurology 2010 10 92 1-11 report about the effect of duration of postherpetic neuralgia on efficacy analyses in a multicenter randomized controlled study of NGX-4010, an 8% capsaicin patch evaluated for the treatment of postherpetic neuralgia. L. R. Webster et al., The Journal of Pain, 2010 11(10) 972-982 report about a multicenter randomized double-blind controlled dose finding study of NGX-4010, a high-concentration capsaicin patch, for the treatment of postherpetic neuralgia. G. A. Irving et al., Pain Medicine 2011 12 99-109 relates to a multicenter randomized double-blind controlled study of NGX-4010, a high-concentration capsaicin patch for the treatment of postherpetic neuralgia. L. R. Webster et al., BMC Anesthesiology 2011 11 25 1-8 report about tolerability of NGX-410, a capsaicin 8% dermal patch, following pretreatment with lidocaine 2.5%/prilocaine 2.5% cream in patients with postherpetic neuralgia. M. M. Backonja et al., Pain Med. 2010 11(4):600-8 report about NGX-4010, a high-concentration capsaicin patch, for the treatment of postherpetic neuralgia: a randomized, double-blind, controlled study with an open-label extension.

C. Maihöfner et al., CMRO 2013 29(6) 673-683 report about the first results of a prospective non-interventional study on the tolerability and analgesic effectiveness over 12 weeks after a single application of capsaicin 8% cutaneous patch in 1044 patients with peripheral neuropathic pain (QUEPP study). C. Maihöfner et al., Eur J Pain 2014 18 671-679 relates to the impact of pre-existing pain in the QUEPP-study on treatment of peripheral neuropathic pain by topical capsaicin.

C. Mankowski et al., BMC neurology 2017 17 80 2-11 report about the effectiveness of the capsaicin 8% patch in the management of peripheral neuropathic pain in European clinical practice (ASCEND study).

It has been reported that the 8% capsaicin patch is particularly useful in the treatment of refractory neuropathic pain syndromes. M. Giménez-Millà et al. BMC Anesthesiology 2014 14 120 1-7 report about the assessment of the feasibility of high-concentration capsaicin patches in the pain unit of a tertiary hospital for a population of mixed refractory peripheral neuropathic pain syndromes in non-diabetic patients. P. Bardo-Brouard et al., Curr Med Res Opin 2018 34(5) 887-891 report about a case series regarding high-concentration topical capsaicin in the management of refractory neuropathic pain in patients with neurofibromatosis type 1.

Long-term treatment and repeated treatments with high-concentration capsaicin patches have been investigated in open label extension trials to the randomized controlled clinical trials and in two larger stand-alone clinical trials. D. M. Simpson et al., J Pain Sympt Manag 2010 39(6) 1053-1064 report about long-term safety of NGX-4010, a high-concentration capsaicin patch, in patients with peripheral neuropathic pain. A previous investigation of repeated applications of high-concentration capsaicin patches in a randomized controlled clinical trial of 52 weeks duration in patients with painful diabetic peripheral neuropathy has shown that high-concentration capsaicin patch on top of standard of care treatment demonstrated greater efficacy than standard of care alone (PACE study, A. I. Vinik et al., BMC Neurology 2016 16 251 1-14). Likewise in a 52 week trial in non-diabetic peripheral neuropathic pain patients in the subset of patients who received 4 consecutive capsaicin 8% patch treatments (n=100), a reduction in average daily pain was observed after each successive capsaicin and/or capsaicinoid treatment, and pain relief was sustained between treatments (STRIDE study, R. Gälvez et al. Clin J Pain 2017 33(10) 921-931).

A problem of conventional treatment of neuropathic pain by topical application of high-concentration capsaicin patches has been that a considerable number of patients do not respond. N. P. Katz et al., Clin J Pain 2015 31(10) 859-866 report about predictors of response in patients with postherpetic neuralgia and HIV-associated neuropathy treated with the 8% capsaicin patch (Qutenza®). Ch. Martini et al., Journal of Pain Research 2012 5 51-59 report that treatment of chronic pain is associated with high variability in the response to pharmacological interventions. A mathematical pharmacodynamic model was developed to quantify the magnitude and onset/offset times of effect of a single capsaicin 8% patch application in the treatment of painful diabetic peripheral neuropathy in 91 patients. The model identified four distinct subgroups that responded differently to treatment: 3.3% of patients (subgroup 1) showed worsening of pain; 31% (subgroup 2) showed no change; 32% (subgroup 3) showed a quick reduction in pain that reached a nadir in week 3, followed by a slow return towards baseline (16%±6% pain reduction in week 12); 34% (subgroup 4) showed a quick reduction in pain that persisted (70%±5% reduction in week 12). The analysis allowed separation of a heterogeneous neuropathic pain population into four homogenous subgroups with distinct behaviors in response to treatment with capsaicin.

Thus, there is a considerable number of patients who do not properly respond to topical treatment with high-concentration capsaicin (e.g. 8% capsaicin patch or cream) such that treatment is terminated. Available data and literature do not provide detailed insights into responder rates beyond the first application of high-concentration capsaicin patch.

There is a demand for medicaments that are useful for the treatment of neuropathic conditions, preferably neuropathic pain, more preferably peripheral neuropathic pain, that overcome the drawbacks of the prior art. In particular, it would be desirable to make available topical treatment of peripheral neuropathic pain by means of capsaicin also to patients who do not initially respond to high-concentration capsaicin topical dose units such as high-concentration topical patch or cream.

It is an object of the invention to provide medicaments that are useful in the treatment of pain, preferably in the topical treatment of neuropathic pain, and that have advantages compared to the prior art.

This object has been achieved by the subject-matter of the patent claims.

### SUMMARY OF THE INVENTION

A first aspect of the invention relates to
one or more high-concentration capsaicin and/or capsaicinoid topical dose units comprising capsaicin and/or capsaicinoid at a concentration of at least 2.5 wt.-%, preferably at least 5 wt.-%, relative to the total weight of dose unit; preferably one or more high-concentration capsaicin and/or capsaicinoid pharmaceutical patches comprising capsaicin and/or capsaicinoid at a concentration of at least 2.5 wt.-%, preferably at least 5 wt.-%, relative to the total weight of the patch without release liner;
for use in the treatment of a neuropathic condition, preferably neuropathic pain,
in a patient who upon previous topical administration of capsaicin and/or capsaicinoid,
preferably by means of one or more high-concentration capsaicin and/or capsaicinoid topical dose units comprising capsaicin and/or capsaicinoid at a concentration of at least 2.5 wt.-%, preferably at least 5 wt.-%, relative to the total weight of dose unit; preferably one or more high-concentration capsaicin and/or capsaicinoid pharmaceutical patches comprising capsaicin and/or capsaicinoid at a concentration of at least 2.5 wt.-%, preferably at least 5 wt.-%, relative to the total weight of the patch without release liner;
did not perceive pain relief by at least 30% versus baseline, preferably on the NRS pain rating scale.

In preferred embodiments, the invention relates to
one or more high-concentration capsaicin topical dose units comprising capsaicin at a concentration of at least 2.5 wt.-%, preferably at least 5 wt.-%, relative to the total weight of dose unit; preferably one or more high-concentration capsaicin pharmaceutical patches comprising capsaicin at a concentration of at least 2.5 wt.-%, preferably at least 5 wt.-%, relative to the total weight of the patch without release liner;
for use in the treatment of a neuropathic condition, preferably neuropathic pain,
in a patient who upon previous topical administration of capsaicin,
preferably by means of one or more high-concentration capsaicin topical dose units comprising capsaicin at a concentration of at least 2.5 wt.-%, preferably at least 5 wt.-%, relative to the total weight of dose unit; preferably one or more high-concentration capsaicin pharmaceutical patches comprising capsaicin at a concentration of at least 2.5 wt.-%, preferably at least 5 wt.-%, relative to the total weight of the patch without release liner;
did not perceive pain relief by at least 30% versus baseline, preferably on the NRS pain rating scale.

The one or more high-concentration capsaicin and/or capsaicinoid topical dose units (topical systems) according to the invention are for use in the treatment of a neuropathic condition, preferably neuropathic pain, more preferably peripheral neuropathic pain. Further preferred aspects of treating neuropathic conditions according to the invention involve regenerating and/or restoring sensory nerve fibers, conversion of non-responders with respect to previous topical administration of capsaicin and/or capsaicinoid into a responders, increasing pain relief to more than 30% versus baseline, and the like.

It has been surprisingly found that by continued (e.g. repeated) topical administration of capsaicin to patients who did not or did not sufficiently respond to initial topical administration of capsaicin (initial non-responders), these patients become responsive to capsaicin treatment over time, i.e. are converted from initial non-responders to responders. Thus, it has been surprisingly found that in spite of initial non-responsiveness, topical administration of capsaicin should be continued thereby achieving improvement with regard to the neuropathic condition the patient suffers from, especially achieving relief of neuropathic pain.

Repeat application also to initial non-responders provides additional benefit to patients. Across trials, more than one third of initial non-responders converted into responders upon repeated treatment with high-concentration capsaicin dose units (patches). Although at month 12, the responder rates in patients who initially did not or did not sufficiently respond to high-concentration capsaicin and/or capsaicinoid dose units (patches) were approximately 10% lower than in the overall population, the substantial increase in responder rates in those who initially did not respond, justifies a repeat treatment.

Furthermore, additional data shows that patients who have a lower initial response and receive a second application sooner (i.e. with a treatment interval <84 days), will proportionally benefit more from a 2nd application than those who had a better initial response and were re-treated later.

In order for capsaicin to exert its effect, it is assumed that the TRPV1 receptor is functioning. If nerves are not expressing functional TRPV1 receptors logically capsaicin cannot exert its effect. It can therefore be reasonably assumed that in a subset of patients that is not responding to capsaicin this receptor is not functioning sufficiently well to generate a response. As a result it is surprising that nerves not expressing sufficiently functioning TRPV1 receptors, can turn into nerves sufficiently expressing functioning TRPV1 receptors after a first or second application of capsaicin even if this application was not successful during a first or second administration to activate the receptors in such a way that a clinical response could be obtained.

### DETAILED DESCRIPTION OF THE INVENTION

The one or more high-concentration capsaicin and/or capsaicinoid topical dose units, preferably high-concentration capsaicin and/or capsaicinoid pharmaceutical patches for use according to the invention contain capsaicin {i.e. (E)-8-methyl-N-vanillyl-6-nonenamide, trans-8-methyl-N-vanillyl nonenamide, 6-nonenamide, (E)-N-[(4-hydroxy-3-methoxyphenyl) methyl]-8-methyl} and/or one or more capsaicinoids. Capsaicin itself is sometimes regarded as a capsaicinoid. For the purpose of the specification, however, capsaicin is no capsaicinoid.

For the purpose of the specification “capsaicin and/or capsaicinoid” means either (i) capsaicin or (ii) capsaicinoid or (iii) capsaicin and capsaicinoid, whereas in each case capsaicinoid may be a single capsaicinoid or any combination of capsaicinoids with one another. Unless expressly stated otherwise, all weights and percentages refer to the total amount of all capsaicin and/or capsaicinoid that is present.

Capsaicinoids are known to the skilled person and commercially available. Preferred capsaicinoids according to the invention include but are not limited to zucapsaicin (cis-capsaicin, Civamide), (6,7-)dihydrocapsaicin, norcapsaicin, nordihydrocapsaicin I, nordihydrocapsaicin II, homocapsaicin I, homocapsaicin II, homodihydrocapsaicin I, homodihydrocapsaicin II, nornorcapsaicin, nornordihydrocapsaicin, octanoyl vanillylamide, nonanoyl vanillylamide (Nonivamide), decanoyl vanillylamide, and mixtures thereof.

Capsaicin and capsaicinoids are N-acyl derivatives of vanillylamine having different acyl chains R, as shown here below:

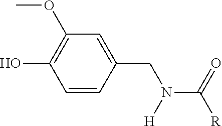

| Trivial names | R = |
|---|---|
| Capsaicin (trans) | —(E)—(CH₂)₄—CH═CH—CH(CH₃)₂ |
| Zucapsaicin, cis-capsaicin, | —(Z)—(CH₂)₄—CH═CH—CH(CH₃)₂ |
| Civamide | |
| (6,7-)Dihydrocapsaicin | —(CH₂)₆—CH(CH₃)₂ |
| Norcapsaicin | —(CH₂)₃—CH═CH—CH(CH₃)₂ |
| Nordihydrocapsaicin I | —(CH₂)₅—CH(CH₃)₂ |
| Nordihydrocapsaicin II | —(CH₂)₄—CH(CH₃)—CH₂CH₃ |
| (anteiso) | |
| Homocapsaicin I | —(CH₂)₄—CH═CH—CH₂CH(CH₃)₂ |
| Homocapsaicin II (anteiso) | —(CH₂)₄—CH═CH—CH(CH₃)—CH₂CH₃ |
| Homodihydrocapsaicin I | —(CH₂)₇—CH(CH₃)₂ |
| Homodihydrocapsaicin II | —(CH₂)₆—CH(CH₃)—CH₂CH₃ |
| (anteiso) | |
| Nornorcapsaicin | —(CH₂)₂—CH═CH—CH(CH₃)₂ |
| Nornordihydrocapsaicin | —(CH₂)₄—CH(CH₃)₂ |
| Octanoyl vanillylamide | —(CH₂)₆—CH₃ |
| Nonanoyl vanillylamide, | —(CH₂)₇—CH₃ |
| Nonivamide | |
| Decanoyl vanillylamide | —(CH₂)₈—CH₃ |

In a preferred embodiment, the one or more high-concentration capsaicin and/or capsaicinoid topical dose units, preferably high-concentration capsaicin and/or capsaicinoid pharmaceutical patches for use according to the invention contain capsaicin but essentially no capsaicinoid.

For the purpose of the specification, a “dose unit” is defined as a predetermined quantity of a pharmaceutical formulation containing a high-concentration of capsaicin and/or capsaicinoid for topical application to the skin of a patient.

For example, when the pharmaceutical formulation is a cream provided in a dispensing device (e.g. tube), a dose unit according to the invention may be e.g. a strand of cream of predetermined length taken from the dispensing device. Said strand of cream of predetermined length contains a predetermined dose of capsaicin and/or capsaicinoid which, when the strand of cream is applied to and spread over the painful skin of the patient, is partially administered to the patient. A skilled person recognizes that various topical systems such as creams, gels, ointments and other liquid or semisolid formulations may be applied to the skin at various thicknesses so that the applied dose per area of skin, e.g. expressed in terms of μg/cm², may vary. However, as the dose unit is typically applied to the skin for a comparatively short application period of time before it is removed, e.g. 15 to 90 minutes, preferably 30 to 60 minutes, these potential variations in thickness will typically not significantly alter the administered dose of capsaicin and/or capsaicinoid. Typically, within such comparatively short application periods, less than 100% of the capsaicin and/or capsaicinoid that was originally contained in the dose units will be administered, whereas a significant quantity of capsaicin and/or capsaicinoid will be removed along with the remainder of the dose unit at the end of the application period.

Likewise, when the pharmaceutical formulation is provided in form of a topical patch, a dose unit according to the invention may be a patch that is optionally cut to match the size and shape of the treatment area. Said patch contains a predetermined dose of capsaicin and/or capsaicinoid which, when the patch is applied to the painful skin of the patient, is partially administered to the patient.

Irrespective of the type of pharmaceutical formulation (e.g. cream, gel, ointment, or patch), the one or more topical dose units according to the invention contain a comparatively high-concentration of capsaicin and/or capsaicinoid of at least 2.5 wt.-%, preferably at least 5 wt.-%, preferably at least 6.0 wt.-%, more preferably at least 7.0 wt.-%, most preferably at least or about 8.0 wt.-%, relative to the total weight of the dose units.

For the purpose of the specification, unless expressly stated otherwise, “high-concentration capsaicin and/or capsaicinoid topical dose unit” refers to a dose unit comprising capsaicin and/or capsaicinoid at a concentration of at least 2.5 wt.-%, preferably at least 5 wt.-%, preferably at least or about 8 wt.-%, relative to the total weight of dose unit and relative to the total amount of capsaicin and/or capsaicinoid.

Preferably, the high-concentration capsaicin and/or capsaicinoid topical dose unit according to the invention contains capsaicin and/or capsaicinoid in an amount such that when the topical dose unit is applied to the skin of the patient in the prescribed manner, the applied area concentration is at least 400 micrograms of capsaicin and/or capsaicinoid per cm² of topical dose unit, more preferably at least 500 micrograms of capsaicin and/or capsaicinoid per cm² of topical dose unit, still more preferably at least 600 micrograms of capsaicin and/or capsaicinoid per cm² of topical dose unit, most preferably at least or about 640 micrograms of capsaicin and/or capsaicinoid per cm² of topical dose unit.

A high-concentration capsaicin and/or capsaicinoid topical dose unit according to the invention is not particularly limited and can be any topical system.

According to Annex A of FDA Guidance for Industry, Product Title and Initial U.S. Approval in the Highlights of Prescribing Information for Human Prescription Drug and Biological Products—Content and Format, January 2018, the term “system” is used for a drug-containing delivery system that controls the release rate of the drug product from the system by diffusion kinetics, active transport, or other means. The activity is defined in terms of the release rate of the active ingredient(s) from the system over a stated period of time. The rate of release and the total duration of drug release typically appear on the drug product and on the container label and carton labeling, but not on the product title line. Exemplified systems include intrauterine systems, ocular systems, oral mucosal systems, periodontal systems, topical systems, transdermal systems, iontophoretic transdermal systems, and vaginal systems. The high-concentration capsaicin and/or capsaicinoid topical dose unit according to the invention is preferably a “topical system” according to this meaning.

Preferred topical dose units (topical systems) according to the invention include but are not limited to patches (e.g. matrix patches, drug-in adhesive patches, iontophoresis systems), solutions, suspensions, lotions, liniments, creams, ointments, salves, pastes, gels (e.g. hydrogels, lipogels, poly(vinyl alcohol) semi-solid hydrogels), sprays, aerosols, foams, liposome formulations (e.g. liposome systems, liposphere systems, niosomal formulations, drug-in-cyclodextrin-in-deformable liposomes), nanostructured formulations (e.g. nanostructured lipid carrier (NLC)-based gels, nanoemulgels), biodegradable drug platforms (e.g. composed of chitosan and guar gum), and the like.

In preferred embodiments, the high-concentration capsaicin and/or capsaicinoid topical dose unit according to the invention is selected from aerosols (i.e. topical aerosols), creams, foams (i.e. topical foams), gels (i.e. topical gels), lotions, ointments, pastes, powders (i.e. topical powders), solutions (i.e. topical solutions), sprays (i.e. topical sprays), suspensions (i.e. topical suspensions), swabs, and systems (i.e. topical systems); preferably all in accordance with Annex A of FDA Guidance for Industry, Product Title and Initial U.S. Approval in the Highlights of Prescribing Information for Human Prescription Drug and Biological Products—Content and Format, January 2018.

The high-concentration capsaicin and/or capsaicinoid topical dose units according to the invention may be provided in form of application aids such as impregnated gauzes, impregnated wipes, impregnated sponges, impregnated fabrics (e.g. woven, non-woven, knit). The high-concentration capsaicin and/or capsaicinoid topical dose units according to the invention may be provided in form of application devices or dispensing devices such as spray dispenser, foam dispenser, sticks, roll-ons, smooth-ons, and the like. Low-concentration capsaicin roll-ons are commercially available e.g. under the tradename arth R_{X}®, Reliaderm®, mintedLeaf.

Preferred high-concentration capsaicin and/or capsaicinoid topical dose units according to the invention comprise capsaicin and/or capsaicinoid and one or more additives selected from hyaluronic acids, surfactants, penetration enhancers, alcohols. Topical dose units of this type are known from U.S. Pat. Nos. 9,956,190, 10,085,956, 10,206,892, and 10,583,100. Preferably, the hyaluronic acid is a mixture of two hyaluronic acids having different molecular weights, preferably high and low. Preferably, the surfactant is a nonionic surfactant, wherein the nonionic surfactant is preferably selected from polysorbates such as polysorbate 80, Cremophor® RH 40 (polyoxy 40 hydrogenated castor oil), sorbitan esters (Spans), poloxamers, cetyl alcohol, cetostearyl alcohol, polyethoxylated alcohols, polyoxyethylene sorbitan, octoxynol, stearyl alcohol etc. Polysorbate 80 (PS 80) and polyox 40 hydrogenated castor oil are particularly preferred. Preferably, the penetration enhancer is selected from glycol monoethyl ether (DGME), propylene glycol, ethoxydiglycol, and dimethyl isosorbide. DGME and propylene glycol are particularly preferred. Preferably, the alcohol is selected from ethyl alcohol, benzyl alcohol, glycerol, propanol, isopropyl alcohol, polyethylene glycol, polyethylene glycols, etc. Ethyl alcohol (ethanol) is particularly preferred.

Preferably, the high-concentration capsaicin and/or capsaicinoid topical dose unit according to the invention in each case is a high-concentration capsaicin and/or capsaicinoid pharmaceutical patch comprising capsaicin and/or capsaicinoid at a concentration of at least 2.5 wt.-%, preferably at least 5 wt.-%, preferably at least 6.0 wt.-%, more preferably at least 7.0 wt.-%, most preferably at least or about 8.0 wt.-%, relative to the total weight of the patch without release liner.

For the purpose of the specification, unless expressly stated otherwise, “high-concentration capsaicin and/or capsaicinoid pharmaceutical patch” refers to a pharmaceutical patch comprising capsaicin and/or capsaicinoid at a concentration of at least 2.5 wt.-%, preferably at least 5 wt.-%, preferably at least or about 8 wt.-%, relative to the total weight of the patch without release liner.

Preferably, the one or more high-concentration capsaicin and/or capsaicinoid pharmaceutical patches each comprise a backing layer, an adhesive layer, and a release liner; and wherein the content of capsaicin and/or capsaicinoid is at least 2.5 wt.-%, preferably at least 5 wt.-%, more preferably at least 6.0 wt.-%, still more preferably at least 7.0 wt.-%, most preferably at least or about 8.0 wt.-%, relative to the total weight of a pharmaceutical patch without release liner. Preferably, the capsaicin and/or capsaicinoid is contained in the adhesive layer (drug-in-adhesive).

For the purpose of the specification, a high-concentration capsaicin and/or capsaicinoid pharmaceutical patch comprising a backing layer, an adhesive layer, and a release liner; wherein the content of capsaicin and/or capsaicinoid is about 8.0 wt.-%, relative to the total weight of a pharmaceutical patch without release liner, is also referred to as “8% capsaicin and/or capsaicinoid patch”.

Preferably, the one or more high-concentration capsaicin and/or capsaicinoid pharmaceutical patches are cutaneous patches containing at least 400 micrograms of capsaicin and/or capsaicinoid per cm² of patch, more preferably at least 500 micrograms of capsaicin and/or capsaicinoid per cm² of patch, still more preferably at least 600 micrograms of capsaicin and/or capsaicinoid per cm² of patch, most preferably at least or about 640 micrograms of capsaicin and/or capsaicinoid per cm² of patch.

Preferably, the one or more high-concentration capsaicin and/or capsaicinoid pharmaceutical patches are cutaneous patches each containing 640 micrograms of capsaicin and/or capsaicinoid per cm² of patch and having an area of 280 cm² (14 cm×20 cm) such that each patch contains a total of 179 mg of capsaicin and/or capsaicinoid. Preferably, each high-concentration capsaicin and/or capsaicinoid pharmaceutical patch consists of an adhesive side containing the active substance and an outer surface backing layer. The adhesive side is preferably covered with a removable release liner. Preferably, the adhesive side is composed of a matrix comprising capsaicin and/or capsaicinoid, silicone adhesives, diethylene glycol monoethyl ether, silicone oil and ethylcellulose. Preferably, the surface backing layer is composed of a polyethylene terephthalate film with siliconized inner side. Preferably, the removable protective layer (release liner) is composed of a polyester film coated with a fluoropolymer. High-concentration capsaicin pharmaceutical patches of this type (8% capsaicin patches) are commercially available under the tradename Qutenza®.

Preferably, the one or more high-concentration capsaicin and/or capsaicinoid topical dose units (topical systems) according to the invention, preferably one or more high-concentration capsaicin and/or capsaicinoid pharmaceutical patches, are not permanently applied to the skin of the patient but only for a comparatively short application period that is needed in order to topically administer capsaicin and/or capsaicinoid from the one or more dose units and patches, respectively, into the patient's skin. Administration is topical, penetration of capsaicin and/or capsaicinoid is intradermally and preferably not systemically.

The one or more high-concentration capsaicin and/or capsaicinoid topical dose units (topical systems) according to the invention, preferably one or more high-concentration capsaicin and/or capsaicinoid pharmaceutical patches, are preferably for single use.

When the one or more high-concentration capsaicin and/or capsaicinoid topical dose units (topical systems) according to the invention are provided in form of one or more high-concentration capsaicin and/or capsaicinoid pharmaceutical patches, said patches are preferably cut to match the size and shape of the treatment area. Preferably, the one or more high-concentration capsaicin and/or capsaicinoid pharmaceutical patches are cut prior to removal of the release liner. When treating feet, the one or more high-concentration capsaicin and/or capsaicinoid pharmaceutical patches are preferably wrapped around the dorsal, lateral and plantar surfaces of each foot to completely cover the treatment area.

Preferably, the one or more high-concentration capsaicin and/or capsaicinoid topical dose units (topical systems) according to the invention, preferably one or more high-concentration capsaicin and/or capsaicinoid pharmaceutical patches, are applied to the skin of the patient for an application period of 15 to 90 minutes, preferably 30 to 60 minutes, and subsequently removed.

Preferably, the one or more high-concentration capsaicin and/or capsaicinoid topical dose units (topical systems) according to the invention, preferably one or more high-concentration capsaicin and/or capsaicinoid pharmaceutical patches, are applied to the most painful skin areas (using up to a maximum of 4 high-concentration capsaicin and/or capsaicinoid dose units and high-concentration capsaicin and/or capsaicinoid pharmaceutical patches, respectively). For the purpose of the specification, applying “one or more high-concentration capsaicin and/or capsaicinoid topical dose units, preferably one or more high-concentration capsaicin and/or capsaicinoid pharmaceutical patches to the skin” refers to simultaneous or essentially simultaneous use of 1, 2, 3 or 4 high-concentration capsaicin and/or capsaicinoid dose units and high-concentration capsaicin and/or capsaicinoid pharmaceutical patches, respectively, preferably 1, 2, 3 or 4 8% capsaicin and/or capsaicinoid patches. More than a single high-concentration capsaicin and/or capsaicinoid pharmaceutical patch may be needed because the area of the skin of the patient to be covered with a high-concentration capsaicin and/or capsaicinoid pharmaceutical patch is larger than can be covered with a single high-concentration capsaicin and/or capsaicinoid pharmaceutical patch.

The painful area is preferably determined by the physician and marked on the skin. The one or more high-concentration capsaicin and/or capsaicinoid topical dose units (topical systems) according to the invention, preferably one or more high-concentration capsaicin and/or capsaicinoid pharmaceutical patches, are preferably applied to intact, non-irritated, dry skin, and allowed to remain in place for an application period of 15 to 45 minutes, preferably 30 minutes for the feet (e.g. for treating HIV-associated neuropathy, painful diabetic peripheral neuropathy) and for an application period of 45 to 75 minutes, preferably 60 minutes for other locations (e.g. for treating postherpetic neuralgia). In case of high-concentration pharmaceutical patches, they are preferably first cut to match the size and form of the painful area of the skin, and subsequently applied to the skin.

For the purpose of the specification, unless expressly stated otherwise, “application period” preferably refers to a period of 15 to 90 minutes, preferably 30 to 60 minutes, during which the one or more high-concentration capsaicin and/or capsaicinoid topical dose units (topical systems) according to the invention, preferably one or more high-concentration capsaicin and/or capsaicinoid pharmaceutical patches are applied to the skin of the patient before they are removed.

Application periods shorter than 15 minutes and longer than 90 minutes are also contemplated according to the invention.

When the concentration of capsaicin and/or capsaicinoid in the high-concentration capsaicin and/or capsaicinoid topical dose unit (topical systems) according to the invention is particularly high, e.g. greater than 8 wt.-%, relative to the total weight of the dose unit, application periods shorter than 15 minutes may be sufficient in order to topically administer the desired dose of capsaicin and/or capsaicinoid.

When the concentration of capsaicin and/or capsaicinoid in the high-concentration capsaicin and/or capsaicinoid topical dose unit (topical systems) according to the invention is particularly low, e.g. below 8 wt.-%, relative to the total weight of the dose unit, application periods longer than 90 minutes may be required in order to topically administer the desired dose of capsaicin and/or capsaicinoid. Under these circumstances, it is also contemplated to apply several high-concentration capsaicin and/or capsaicinoid topical dose units (topical systems) according to the invention having a capsaicin and/or capsaicinoid concentration below 8 wt.-%, relative to the total weight of the dose unit, repeatedly on several consecutive days, each dose unit for an application period of 15 to 90 minutes, preferably 30 to 60 minutes. For example, it is contemplated to apply assigned doses for 60 minutes on each of four consecutive days.

The capsaicin and/or capsaicinoid contained in the high-concentration topical dose unit (topical system) according to the invention is intended for delivery into the skin. For any high-concentration topical dose unit the administered dose of capsaicin and/or capsaicinoid from the high-concentration topical dose unit (topical system) is a function of the application period (application time), whereas depending upon the individual formulation, the rate of release may be linear of change over time. The individual rate of release and other pharmacokinetic parameters can be determined by routine experiments that are well acknowledged in the art (active substance dissolution and skin permeation assays). For example, based on in vitro studies with Qutenza® (capsaicin concentration 8 wt.-%, 640 μg of capsaicin per cm² of patch), approximately 1% of capsaicin is estimated to be absorbed into the epidermal and dermal layers of skin during one-hour applications (i.e. about 6.4 μg·cm⁻²).

Preferably, the application period is adjusted such that depending upon the individual properties of the high-concentration topical dose unit (type, concentration of capsaicin and/or capsaicinoid, excipients such as penetration enhancers, rate of release, etc.) the dose of capsaicin and/or capsaicinoid per area of skin that is actually administered during the application period is at least 4.0 μg·cm⁻², more preferably at least 4.5 μg·cm⁻², still more preferably at least 5.0 μg·cm⁻², even more preferably at least 5.5 μg·cm⁻², most preferably at least 6.0 μg·cm⁻².

After removal of the one or more high-concentration capsaicin and/or capsaicinoid topical dose units, preferably one or more high-concentration capsaicin and/or capsaicinoid pharmaceutical patches from the skin, a cleansing gel is preferably applied liberally to the treatment area and left on for at least one minute. The cleansing gel is then preferably be wiped off with dry gauze to remove any remaining capsaicin and/or capsaicinoid from the skin. After the cleansing gel has been wiped off, the area of the skin is preferably gently washed with soap and water. A suitable cleansing gel may contain macrogol 300, carbomer, purified water, sodium hydroxide, disodium edetate, and butyl hydroxy anisole.

Preferably, the one or more high-concentration capsaicin and/or capsaicinoid topical dose units (topical systems) according to the invention, preferably one or more high-concentration capsaicin and/or capsaicinoid pharmaceutical patches, are applied to the skin of the patient, remain on the skin for an application period thereby topically administering capsaicin and/or capsaicinoid during the application period, and are subsequently removed, whereby as a consequence of topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient preferably perceives pain relief by at least 30% versus baseline, on either the VAS or the NRS pain rating scale, preferably the NRS pain rating scale. It may take between 1 to 3 weeks from the application period until onset of analgesia. Further, it may take repeated application of one or more high-concentration capsaicin and/or capsaicinoid topical dose units (topical systems) according to the invention, preferably one or more high-concentration capsaicin and/or capsaicinoid pharmaceutical patches, until an initial non-responder converts into a responder.

After application periods of e.g. 15 to 90 minutes, preferably 30 to 60 minutes, topical administration has been effected so that the one or more high-concentration capsaicin and/or capsaicinoid topical dose units (topical systems) according to the invention, preferably one or more high-concentration capsaicin and/or capsaicinoid pharmaceutical patches, are preferably removed from the skin. When proceeding this way, after removal from the skin, as a consequence of the administered dose of capsaicin and/or capsaicinoid, treatment of the neuropathic condition such as pain relief may last for up to 90 days or even longer.

Preferably, the one or more high-concentration capsaicin and/or capsaicinoid topical dose units (topical systems) according to the invention, preferably one or more high-concentration capsaicin and/or capsaicinoid pharmaceutical patches, are applied to the skin of the patient, remain on the skin for an application period thereby topically administering capsaicin and/or capsaicinoid during the application period, and are subsequently removed, whereby as a consequence of topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient preferably perceives pain relief by at least 30% versus baseline for a period of at least 4 weeks, more preferably at least 6 weeks, still more preferably at least 8 weeks. As it may take between 1 to 3 weeks from the application period until onset of analgesia, the period of pain relief does not necessarily commence immediately after the application period.

Subsequent application of one or more another high-concentration capsaicin and/or capsaicinoid dose units according to the invention, preferably one or more another high-concentration capsaicin and/or capsaicinoid pharmaceutical patches, may be preferably repeated after 2 to 4 months, preferably after 2 months or after 3 months, e.g. every 60 days or every 90 days.

In a preferred embodiment, subsequent application of one or more another high-concentration capsaicin and/or capsaicinoid dose units according to the invention, preferably one or more another high-concentration capsaicin pharmaceutical patches, is repeated after 2 to 3 months, preferably after less than 90 days, more preferably after less than 84 days, still more preferably after 2 months, e.g. every 60 days. It has been surprisingly found that the response rate can be relatively increased when subsequent application of one or more another high-concentration capsaicin and/or capsaicinoid dose units according to the invention proceeds in shorter intervals, preferably not later than 89 days, or not later than 88 days, or not later than 87 days, or not later than 86 days, or not later than 85 days, or not later than 84 days, or not later than 83 days, or not later than 82 days, or not later than 81 days; more preferably not later than 80 days, or not later than 79 days, or not later than 78 days, or not later than 77 days, or not later than 76 days, or not later than 75 days, or not later than 74 days, or not later than 73 days, or not later than 72 days, or not later than 71 days; still more preferably not later than 70 days, or not later than 69 days, or not later than 68 days, or not later than 67 days, or not later than 66 days, or not later than 65 days, or not later than 64 days, or not later than 63 days, or not later than 62 days, or not later than 61 days, or not later than 60 days, in each case after the previous topical administration of capsaicin and/or capsaicinoid.

In a particularly preferred embodiment, treatments may be repeated by subsequent application every 90 days, as warranted by the persistence or return of pain. Re-treatment after less than 90 days is preferably considered for individual patients. A minimum interval of 60 days between treatments is preferably observed.

For the purpose of the specification, two different preferred administration regimens are distinguished:
according to a relatively fast administration regimen, hereinafter also referred to as “[f]”, the time span between two consecutive application periods is less than 90 days, preferably less than 84 days, more preferably about 2 months, e.g. 60 days;
according to a relatively slow administration regimen, hereinafter also referred to as “[s]”, the time span between two consecutive application periods is about 3 months, e.g. 90 days.

It is also contemplated according to the invention that the time span between two consecutive application periods may vary, especially may be increased in the course of the overall treatment period. For example, in a preferred embodiment, the time span between the first application period and the second application period is less than 90 days, preferably less than 84 days, more preferably about 2 months, e.g. 60 days, whereas the subsequent time span between the second application period and the third application period is about 3 months, e.g. 90 days.

It is also contemplated according to the invention that the time span between two consecutive application periods may be determined by the patient in view of the individual pain perception.

When application is repeated, said one or more another high-concentration capsaicin and/or capsaicinoid dose units, preferably high-concentration capsaicin and/or capsaicinoid pharmaceutical patches, are preferably applied to the same painful area of the skin of the patient where the one or more high-concentration capsaicin and/or capsaicinoid topical dose units (topical systems), preferably one or more high-concentration capsaicin and/or capsaicinoid pharmaceutical patches, were also previously applied, i.e. 2 to 4 months ago.

Although currently there is no restriction on use of high-concentration topical capsaicin and/or capsaicinoid also in those patients not initially responding to treatment, no improvement with repeated treatment in this subgroup of patients has been demonstrated so far.

It is contemplated according to the invention that the treatment area of the painful skin of the patient may be pre-treated with a topical anesthetic (e.g. topical lidocaine (4%), or topical lidocaine (2.5%)/prilocaine (2.5%)) or the patient may be administered an oral analgesic (e.g. 50 mg of tramadol) prior to application of the one or more high-concentration capsaicin and/or capsaicinoid topical dose units (topical systems) according to the invention, preferably one or more high-concentration capsaicin and/or capsaicinoid pharmaceutical patches, to reduce potential application related discomfort. The topical anesthetic is preferably applied to cover the entire treatment area and surrounding 1 to 2 cm. Topical anesthetics are preferably removed prior to applying the one or more high-concentration capsaicin and/or capsaicinoid topical dose units, preferably one or more high-concentration capsaicin and/or capsaicinoid pharmaceutical patches.

The one or more high-concentration capsaicin and/or capsaicinoid topical dose units (topical systems) according to the invention, preferably one or more high-concentration capsaicin and/or capsaicinoid pharmaceutical patches, are for use in the treatment of a neuropathic condition in a patient who upon previous topical administration of capsaicin and/or capsaicinoid did not perceive pain relief by at least 30% versus baseline on either the VAS or the NRS pain rating scale, preferably the NRS pain rating scale. Previous topical administration of capsaicin and/or capsaicinoid may principally have been achieved by any suitable pharmaceutical formulation that is capable of topically administering capsaicin and/or capsaicinoid. Preferably, however, previous topical administration of capsaicin and/or capsaicinoid was achieved also by means of one or more high-concentration capsaicin and/or capsaicinoid topical dose units, preferably one or more high-concentration capsaicin and/or capsaicinoid pharmaceutical patches according to the invention as described herein, preferably 8% capsaicin and/or capsaicinoid patches, but was previously not (yet) successful so that the patient did not perceive pain relief by at least 30% versus baseline on either the VAS or the NRS pain rating scale, preferably the NRS pain rating scale. Conventionally, in such a situation, treatment would usually be terminated due to lack of efficiency. According to the invention, however, treatment is continued by again using one or more high-concentration capsaicin and/or capsaicinoid topical dose units (topical systems) according to the invention, preferably one or more high-concentration capsaicin and/or capsaicinoid pharmaceutical patches, preferably 8% capsaicin and/or capsaicinoid patches, thereby converting initial non-responders into responders.

For the purpose of the specification, a patient who upon previous topical administration of capsaicin and/or capsaicinoid did not perceive pain relief by at least 30% versus baseline is also referred to as “initial non-responder”.

As onset of pain relief may occur with a delay after topical administration of capsaicin and/or capsaicinoid by applying one or more high-concentration capsaicin and/or capsaicinoid topical dose units (topical systems) according to the invention, preferably one or more high-concentration capsaicin and/or capsaicinoid pharmaceutical patches, preferably one or more 8% patches, e.g. after 1 to 3 weeks, pain relief versus baseline is preferably determined 4 weeks (28 days) after topical administration.

For the purpose of the specification, consistent with existing literature and guidance, “responders” are defined as those patients who have achieved a 30% decrease from baseline on a numeric pain rating scale (NRS) or a pain visual analogue scale (VAS). Conversely, “non-responders” are defined as those who did not achieve a 30% decrease from baseline on such rating scale. According to the invention, the timepoint for measuring the sustained therapeutic effect of chronic pain treatments can be after 12 weeks. This time point is conventional and in accordance with EMA pain guidance (15 Dec. 2016 EMA/CHMP/970057/2011 Committee for Medicinal Products for Human Use (CHMP)). However it is common to assess the pain ratings more frequently and therefore, the time point when it is assessed whether a patient is a responder or a non-responder is preferably 8 weeks after the preceding topical administration of capsaicin and/or capsaicinoid.

Preferably, with regard to the previous topical administration of capsaicin and/or capsaicinoid, the patient is an initial non-responder selected from complete non-responders and insufficient responders.

For the purpose of the specification, a “complete non-responder” is a patient who upon previous topical administration of capsaicin and/or capsaicinoid did not perceive any pain relief or even experienced a worsening of pain, preferably on the NRS pain rating scale, preferably upon initial topical administration of capsaicin and/or capsaicinoid by means of the one or more high-concentration capsaicin and/or capsaicinoid topical dose units, preferably one or more high-concentration capsaicin and/or capsaicinoid pharmaceutical patches according to the invention as described herein.

For the purpose of the specification, an “insufficient responder” is a patient who upon previous topical administration of capsaicin and/or capsaicinoid perceived pain relief to some extent, but less than 30% versus baseline, preferably on the NRS pain rating scale, preferably upon initial topical administration of capsaicin and/or capsaicinoid by means of the one or more high-concentration capsaicin and/or capsaicinoid topical dose units, preferably one or more high-concentration capsaicin and/or capsaicinoid pharmaceutical patches according to the invention as described herein.

According to the EMA pain Guidance with reference to Koltzenburg M, McMahon S, Tracey I, Turk D C (ed.) Wall and Melzack's Textbook of Pain. 6th edition, Saunders, imprint of Elsevier Ltd., ISBN 978-0-7020-4059-7, pain intensity (PI) is still the key measure of efficacy of an analgesic drug. Among the pain rating scales the Visual analogue scale (VAS), numeric rating scale (NRS) and verbal rating scale (VRS) have been extensively used and validated. The VAS is a continuous variable on a 10 cm line representing “no pain” to “worst imaginable pain”, whereas the NRS is a discrete variable describing pain level with numbers from 0 to 10 (J. T. Farrar et al. Pain 2001; 94:149-58). Due to practical considerations the latter is the most commonly used scale. The VRS, consisting of a series of verbal pain descriptors, has been shown to lack sensitivity to detect changes in PI when compared with VAS or NRS.

For the purpose of the specification, a “responder” is defined as someone who reported a decrease of 30% from baseline on either the VAS or the NRS pain rating scale, preferably the NRS pain rating scale.

The preferred administration regimens defined hereinafter typically involve simultaneous and/or repeated application of one or more high-concentration capsaicin and/or capsaicinoid does units, typically more than a single high-concentration capsaicin and/or capsaicinoid pharmaceutical patch according to the invention. Thus, in order to put these preferred administration regimens into practice, a plurality of high-concentration capsaicin and/or capsaicinoid dose units according to the invention, preferably a plurality of high-concentration pharmaceutical patches are used, preferably a plurality of 8% capsaicin and/or capsaicinoid patches.

In a preferred embodiment, the patient to be treated according to the invention may have a treatment history where
(a-i) one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches, were previously applied to the skin of the patient for a first application period thereby previously topically administering capsaicin and/or capsaicinoid during the first application period, and were subsequently removed, whereby as a consequence of previous topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient did not perceive pain relief by at least 30% versus baseline (initial non-responder).

The previous topical administration under (a-i) is then not part of the use of the one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches. The previous topical administration under (a-i) merely qualifies the patient as an initial non-responder. A skilled person can easily determine in the course of anamnesis whether a patient to be treated is an initial non-responder or not.

Preferably,
(b-i) 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above first application period under (a-i), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a second application period thereby topically administering capsaicin and/or capsaicinoid during the second application period, and are subsequently removed; and
(b-ii) optionally, 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above second application period under (b-i), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a third application period thereby topically administering capsaicin and/or capsaicinoid during the third application period, and are subsequently removed; and
(b-iii) optionally, 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above third application period under (b-ii), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a fourth application period thereby topically administering capsaicin and/or capsaicinoid during the fourth application period, and are subsequently removed; and
(b-iv) optionally, 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above fourth application period under (b-iii), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a fifth application period thereby topically administering capsaicin and/or capsaicinoid during the fifth application period, and are subsequently removed.

In a preferred embodiment, the patient to be treated according to the invention may again have a treatment history where
(a-i) one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches, were previously applied to the skin of the patient for a first application period thereby previously topically administering capsaicin and/or capsaicinoid during the first application period, and were subsequently removed, whereby as a consequence of previous topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient did not perceive pain relief by at least 30% versus baseline (initial non-responder).

The previous topical administration under (a-i) is then again not part of the use of the one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches. The previous topical administration under (a-i) merely qualifies the patient as an initial non-responder. A skilled person can easily determine in the course of anamnesis whether a patient to be treated is an initial non-responder or not.

Preferably,
(b-i) 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above first application period under (a-i), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a second application period thereby topically administering capsaicin and/or capsaicinoid during the second application period, and are subsequently removed;
   preferably whereby as a consequence of topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient preferably perceives pain relief by at least 30% versus baseline (responder), whereas onset of pain relief by at least 30% versus baseline may occur with a delay of 1 to 3 weeks after the second application period; and
(b-ii) optionally, 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above second application period under (b-i), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a third application period thereby topically administering capsaicin and/or capsaicinoid during the third application period, and are subsequently removed;
   preferably whereby as a consequence of topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient preferably still perceives pain relief by at least 30% versus baseline (responder), whereas onset of pain relief by at least 30% versus baseline may occur with a delay of 1 to 3 weeks after the third application period but preferably follows immediately the pain relief by at least 30% versus baseline that was achieved by the above second application period under (b-i).

In a preferred embodiment, the patient to be treated according to the invention may again have a treatment history where
(a-i) one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches, were previously applied to the skin of the patient for a first application period thereby previously topically administering capsaicin and/or capsaicinoid during the first application period, and were subsequently removed, whereby as a consequence of previous topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient did not perceive pain relief by at least 30% versus baseline (initial non-responder).

The previous topical administration under (a-i) is then again not part of the use of the one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches. The previous topical administration under (a-i) merely qualifies the patient as an initial non-responder. A skilled person can easily determine in the course of anamnesis whether a patient to be treated is an initial non-responder or not.

Preferably,
(b-i) 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above first application period under (a-i), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a second application period thereby topically administering capsaicin and/or capsaicinoid during the second application period, and are subsequently removed; and
(b-ii) 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above second application period under (b-i), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a third application period thereby topically administering capsaicin and/or capsaicinoid during the third application period, and are subsequently removed;
   preferably whereby as a consequence of topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient preferably perceives pain relief by at least 30% versus baseline (responder), whereas onset of pain relief by at least 30% versus baseline may occur with a delay of 1 to 3 weeks after the second application period; and
(b-iii) optionally, 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above third application period under (b-ii), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a fourth application period thereby topically administering capsaicin and/or capsaicinoid during the fourth application period, and are subsequently removed;
   preferably whereby as a consequence of topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient preferably still perceives pain relief by at least 30% versus baseline (responder), whereas onset of pain relief by at least 30% versus baseline may occur with a delay of 1 to 3 weeks after the fourth application period but preferably follows immediately the pain relief by at least 30% versus baseline that was achieved by the above third application period under (b-ii).

In a preferred embodiment, the patient to be treated according to the invention may again have a treatment history where
(a-i) one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches, were previously applied to the skin of the patient for a first application period thereby previously topically administering capsaicin and/or capsaicinoid during the first application period, and were subsequently removed, whereby as a consequence of previous topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient did not perceive pain relief by at least 30% versus baseline (initial non-responder).

The previous topical administration under (a-i) is then again not part of the use of the one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches. The previous topical administration under (a-i) merely qualifies the patient as an initial non-responder. A skilled person can easily determine in the course of anamnesis whether a patient to be treated is an initial non-responder or not.

Preferably,
(b-i) 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above first application period under (a-i), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a second application period thereby topically administering capsaicin and/or capsaicinoid during the second application period, and are subsequently removed; and
(b-ii) 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above second application period under (b-i), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a third application period thereby topically administering capsaicin and/or capsaicinoid during the third application period, and are subsequently removed; and
(b-iii) 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above third application period under (b-ii), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a fourth application period thereby topically administering capsaicin and/or capsaicinoid during the fourth application period, and are subsequently removed;
   preferably whereby as a consequence of topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient preferably perceives pain relief by at least 30% versus baseline (responder), whereas onset of pain relief by at least 30% versus baseline may occur with a delay of 1 to 3 weeks after the fourth application period; and
(b-iv) optionally, 2 to 4 months, preferably [f] less than 90 days, more preferably less than 84 days, still more preferably 60 to 83 days, yet more preferably about 2 months, e.g. 60 days; or [s] about 3 months, e.g. 90 days, after the above fourth application period under (b-iii), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a fifth application period thereby topically administering capsaicin and/or capsaicinoid during the fifth application period, and are subsequently removed;
   preferably whereby as a consequence of topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient preferably still perceives pain relief by at least 30% versus baseline (responder), whereas onset of pain relief by at least 30% versus baseline may occur with a delay of 1 to 3 weeks after the fifth application period but preferably follows immediately the pain relief by at least 30% versus baseline that was achieved by the above fourth application period under (b-iii).

In another preferred embodiment, the patient to be treated according to the invention did not perceive pain relief by at least 30% versus baseline upon repeated previous topical administrations of capsaicin and/or capsaicinoid, e.g. after two consecutive previous topical administrations of capsaicin and/or capsaicinoid, or after three consecutive previous topical administrations of capsaicin and/or capsaicinoid (repeated non-responder). Preferably, said repeated previous topical administrations of capsaicin and/or capsaicinoid were also achieved by means of one or more high-concentration capsaicin and/or capsaicinoid dose units according to the invention, preferably one or more high-concentration capsaicin and/or capsaicinoid pharmaceutical patches, more preferably 8% capsaicin and/or capsaicinoid patches, but not successfully so that the patient did previously not perceive pain relief by at least 30% versus baseline, preferably on the NRS pain rating scale.

According to this embodiment, the patient to be treated according to the invention may have a treatment history where
(a-i) one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches, were previously applied to the skin of the patient for a first application period thereby previously topically administering capsaicin and/or capsaicinoid during the first application period, and were subsequently removed, whereby as a consequence of previous topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient did not perceive pain relief by at least 30% versus baseline (initial non-responder); and
(a-ii) 2 to 4 months, preferably [f] less than 90 days, more preferably less than 84 days, still more preferably 60 to 83 days, yet more preferably about 2 months, e.g. 60 days, or [s] 3 months, e.g. 90 days after the above first application period under (a-i), one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches, were previously applied to the skin of the patient for a second application period thereby previously topically administering capsaicin and/or capsaicinoid during the second application period, and were subsequently removed, whereby as a consequence of previous topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient still did not perceive pain relief by at least 30% versus baseline (repeated non-responder).

The above repeated previous topical administrations under (a-i) and (a-ii) are then not part of the use of the one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches. The repeated previous topical administrations under (a-i) and (a-ii) merely qualify the patient as a repeated non-responder. A skilled person can easily determine in the course of anamnesis whether a patient is a repeated non-responder or not.

Preferably,
(b-i) 2 to 4 months, preferably [f] less than 90 days, more preferably less than 84 days, still more preferably 60 to 83 days, yet more preferably about 2 months, e.g. 60 days; or [s] about 3 months, e.g. 90 days, after the second application period under (a-ii), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a third application period thereby topically administering capsaicin and/or capsaicinoid during the third application period, and are subsequently removed;
   preferably whereby as a consequence of topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient preferably perceives pain relief by at least 30% versus baseline (responder), whereas onset of pain relief by at least 30% versus baseline may occur with a delay of 1 to 3 weeks after the second application period; and
(b-ii) optionally, 2 to 4 months, preferably [f] less than 90 days, more preferably less than 84 days, still more preferably 60 to 83 days, yet more preferably about 2 months, e.g. 60 days; or [s] about 3 months, e.g. 90 days, after the third application period under (b-i), one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches, are then applied to the skin of the patient, remain on the skin for a fourth application period thereby topically administering capsaicin and/or capsaicinoid during the fourth application period, and are subsequently removed;
   preferably whereby as a consequence of topical administration of capsaicin and/or capsaicinoid by means of the one or more high-concentration capsaicin and/or capsaicinoid topical dose units, preferably one or more high-concentration capsaicin and/or capsaicinoid pharmaceutical patches, the patient preferably still perceives pain relief by at least 30% versus baseline (responder), whereas onset of pain relief by at least 30% versus baseline may occur with a delay of 1 to 3 weeks after the fourth application period but preferably follows immediately the pain relief by at least 30% versus baseline that was achieved by the above third application period under (b-i).

Preferred administration regimens A to I are compiled in the following table where
one or more high-concentration capsaicin and/or capsaicinoid dose units according to the invention as described herein, preferably one or more high-concentration capsaicin and/or capsaicinoid pharmaceutical patches, more preferably 8% capsaicin and/or capsaicinoid patches, were previously used but had the effect that the patient did not perceive pain relief by at least 30% versus baseline, and
one or more high-concentration capsaicin and/or capsaicinoid dose units according to the invention as described herein, preferably one or more high-concentration capsaicin and/or capsaicinoid pharmaceutical patches, more preferably 8% capsaicin and/or capsaicinoid patches, are being used in the treatment according to the invention with the effect that the patient does perceive pain relief by at least 30% versus baseline:

| | time | | | | | |
|---|---|---|---|---|---|---|
| | patient history to be determined by anamnesis | | | treatment according to the invention | | |
| | previously pain relief <30% versus baseline | | | pain relief ≥30% versus baseline | | |
| | (a-i) | (a-ii) | (a-iii) | (b-i) | (b-ii) | (b-iii) |
| A | X days | — | — | Y days | optionally, Y days | optionally, Y days |
| B | X days | — | — | Y days | Y days | optionally, Y days |
| C | X days | — | — | Y days | Y days | Y days |
| D | X days | X days | — | Y days | optionally, Y days | optionally, Y days |
| E | X days | X days | — | Y days | Y days | optionally, Y days |
| F | X days | X days | — | Y days | Y days | Y days |
| G | X days | X days | X days | Y days | optionally, Y days | optionally, Y days |
| H | X days | X days | X days | Y days | Y days | optionally, Y days |
| I | X days | X days | X days | Y days | Y days | Y days |

In preferred embodiments, when the administration regimen is relatively fast (i.e. [f]), in each case, X is independently of one another 30 to 90, preferably 40 to 80, more preferably 50 to 70, and most preferably about 60. Preferably, X is independently of one another is less than 90, more preferably less than 84, still more preferably 60 to 83. The time intervals of X days immediately follow one another without interruption. Likewise, in each case, Y is independently of one another 30 to 90, preferably 40 to 80, more preferably 50 to 70, and most preferably about 60. The time intervals of Y days immediately follow one another without interruption.

In other preferred embodiments, when the administration regimen is relatively slow (i.e. [s]), in each case, X is independently of one another 60 to 120, preferably 70 to 110, more preferably 80 to 100, and most preferably about 90. The time intervals of X days immediately follow one another without interruption. Likewise, in each case, Y is independently of one another 60 to 120, preferably 70 to 110, more preferably 80 to 100, and most preferably about 90. The time intervals of Y days immediately follow one another without interruption.

The above table provides information about the history of the patient who was previously treated with capsaicin and/or capsaicinoid and is now preferably to be treated according to the invention (patient history, left part of the table, (a-i), (a-ii), and (a-iii), respectively). Previous treatment is not part of the treatment according to the invention but qualifies the patient as initial non-responder or repeated non-responder. With regard to previous treatment, “X days” means that one or more high-concentration capsaicin and/or capsaicinoid topical dose units, preferably one or more high-concentration capsaicin and/or capsaicinoid pharmaceutical patches according to the invention as described herein, more preferably 8% capsaicin and/or capsaicinoid patches, were previously applied to the skin of the patient at the beginning of an interval of X days and remained on the skin for an application period (15 to 90 minutes, preferably 30 to 60 minutes) before they were removed from the skin. During the remainder of the interval of X days, no dose unit or pharmaceutical patch according to the invention as described herein was applied to the skin of the patient. Such previous treatment did not have the desired effect of achieving at least 30% pain relief versus baseline (initial non-responder or repeated non-responder).

Further, the above table provides information about preferred regimens for treating a patient according to the invention (treatment, right part of the table, (b-i), (b-ii), and (b-iii), respectively). With regard to treatment according to the invention, “Y days” means that one or more high-concentration capsaicin and/or capsaicinoid topical dose units, preferably one or more high-concentration capsaicin and/or capsaicinoid pharmaceutical patches according to the invention as described herein, more preferably 8% capsaicin and/or capsaicinoid patches, are to be applied to the skin of the patient in the course of the treatment according to the invention at the beginning of an interval of Y days and remain on the skin for an application period (15 to 90 minutes, preferably 30 to 60 minutes) before they are removed from the skin. During the remainder of the interval of Y days, no dose unit or pharmaceutical patch according to the invention as described herein is applied to the skin of the patient. Such treatment has the desired effect of achieving at least 30% pain relief versus baseline (responder). Further, with regard to treatment according to the invention, “optionally, Y days” means that after treatment under (b-i), i.e. after the 90 days, optionally another treatment by means of one or more high-concentration capsaicin and/or capsaicinoid topical dose units, preferably one or more high-concentration capsaicin and/or capsaicinoid pharmaceutical patches according to the invention as described herein, more preferably 8% capsaicin and/or capsaicinoid patches, may occur but does not have to.

Preferred regimens in accordance with the above table are selected from (a-i)-(b-i), (a-i)-(b-i)(b-ii), (a-i)-(b-i)-(b-ii)-(b-iii); (a-i)-(a-ii)-(b-i), (a-i)-(a-ii)-(b-i)-(b-ii), (a-i)-(a-ii)-(b-i)-(b-ii)-(b-iii); (a-i)(a-ii)-(a-iii)-(b-i), (a-i)-(a-ii)-(a-iii)-(b-i)-(b-ii), and (a-i)-(a-ii)-(a-iii)-(b-i)-(b-ii)-(b-iii).

As already mentioned above, previous topical administration of capsaicin and/or capsaicinoid was preferably achieved by applying one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches, to the skin of the patient for an application period thereby previously topically administering capsaicin and/or capsaicinoid during the application period and subsequently removing said one or more high-concentration capsaicin and/or capsaicinoid topical dose units, preferably one or more high-concentration capsaicin and/or capsaicinoid pharmaceutical patches from the skin. Likewise, repeated previous topical administrations were preferably achieved by repeatedly applying one or more high-concentration capsaicin and/or capsaicinoid topical dose units (topical systems) according to the invention, preferably one or more high-concentration capsaicin and/or capsaicinoid pharmaceutical patches, more preferably 8% capsaicin and/or capsaicinoid patches, to the skin of the patient for an application period thereby previously repeatedly topically administering capsaicin and/or capsaicinoid during the application period and subsequently removing said one or more high-concentration capsaicin and/or capsaicinoid topical dose units, preferably one or more high-concentration capsaicin and/or capsaicinoid pharmaceutical patches from the skin.

Preferably, said one or more high-concentration capsaicin and/or capsaicinoid topical dose units, preferably one or more high-concentration capsaicin and/or capsaicinoid pharmaceutical patches previously used were of the same kind as the one or more high-concentration capsaicin and/or capsaicinoid topical dose units, preferably one or more high-concentration capsaicin and/or capsaicinoid pharmaceutical patches for use according to the invention, more preferably 8% capsaicin and/or capsaicinoid patches.

The invention also contemplates the use of capsaicin and/or capsaicinoid for the manufacture of one or more high-concentration capsaicin and/or capsaicinoid topical dose units, preferably one or more high-concentration capsaicin and/or capsaicinoid pharmaceutical patches comprising capsaicin and/or capsaicinoid, more preferably 8% capsaicin and/or capsaicinoid patches, for treating a neuropathic condition, preferably neuropathic pain, in a patient who upon previous topical administration of capsaicin and/or capsaicinoid did not perceive pain relief by at least 30% versus baseline, preferably on the NRS pain rating scale. Likewise the invention also contemplates a method for treating a neuropathic condition, preferably neuropathic pain, comprising the step of applying one or more high-concentration capsaicin and/or capsaicinoid topical dose units, preferably one or more high-concentration capsaicin and/or capsaicinoid pharmaceutical patches comprising capsaicin and/or capsaicinoid, more preferably 8% capsaicin and/or capsaicinoid patches, to the skin of a patient who upon previous topical administration of capsaicin and/or capsaicinoid did not perceive pain relief by at least 30% versus baseline, preferably on the NRS pain rating scale.

Preferably, the treatment of the neuropathic condition is for relief of neuropathic pain, preferably peripheral neuropathic pain.

Preferably, the neuropathic pain is selected from the group consisting of painful diabetic neuropathy, postherpetic neuralgia, chemotherapy-induced neuropathic pain, HIV-associated neuropathy, small-fiber neuropathy, chronic idiopathic axonal polyneuropathy, post-traumatic neuropathic pain, and post-surgical neuropathic pain.

Preferably, the treatment of the neuropathic condition is for regenerating and/or restoring sensory nerve fibers.

Preferably, the treatment of the neuropathic condition is for converting a non-responder with respect to previous topical administration of capsaicin and/or capsaicinoid into a responder.

Preferably, the treatment of the neuropathic condition is for increasing pain relief to more than 30% versus baseline, preferably at least 60% versus baseline, preferably on the NRS pain rating scale.

### EXAMPLES

The following examples further illustrate the invention but are not to be construed as limiting its scope.

In the two selected trials of 52 week duration, high-concentration capsaicin patch was applied 30 min to the feet or 60 mins to other body areas. All patients were diagnosed with peripheral neuropathic pain. Initial non-responders were defined as patients having a smaller than 30% decrease on the numerical pain rating scale from baseline to 3 months post-baseline. Responders were defined as patients who achieved a ≥30% decrease on the numerical pain rating scale at predefined timepoints (month 6, 9 and 12).

### Example 1

All patients were diagnosed with non-diabetic peripheral neuropathic pain. Repeat treatment with Qutenza® was allowed every 9-12 weeks.

At month 3, 76.6% of patient treated with high-concentration capsaicin patch (n=306) did not meet the responder criteria (n=168). Of these 168, 115 patients (68%) were still in the trial at 12 months. Of note is that from the original trial population, 174 patients (56.9%) were still in the trial at 12 months. Of the initial non-responders (n=115), 57.3% had received at least 4 applications of high-concentration capsaicin patch. Overall at month 12, 33.9% of initial non-responders had become responders to treatment whereas 43.7% of the total population could be classified as responder.

The results are compiled in the tables here below. In each case, only subjects who received the second application are included; * time from the first Qutenza® application to the first Qutenza® reapplication. Summarized is percent change from baseline: if baseline score is 10 and pain decreases to 7 (on the 10 point NRS scale), the change from baseline would be −3 i.e. a % change from baseline of −30%. Mean is the average percent change from baseline for the subjects in the sample. The median is the value that indicates that half of the subjects have a % decrease below and half of the subjects have % decrease above the indicated value. Q1 and Q3 indicate that 25% and 75% of the patients have a value below the % decrease value mentioned respectively). The min-max values are the largest decrease and the smallest decrease (or largest increase if number is positive). The 95% CI is the 95% confidence interval around the mean:

A) Baseline NRS pain rating score:

| | Qutenza ® | Qutenza ® | Qutenza ® | Qutenza ® |
|---|---|---|---|---|
| overall | <84 days* | 84-109 days* | >110 days* | total |
| baseline | n = 111 | n = 67 | n = 52 | n = 230 |
| n | 111 | 66 | 52 | 229 |
| missing | 0 | 1 | 0 | 1 |
| mean (SD) | 6.7 (1.29) | 6.9 (1.37) | 6.4 (1.30) | 6.7 (1.32) |
| median (Q1, Q3) | 6.9 (5.9, 7.7) | 6.9 (6.2, 7.8) | 6.5 (5.5, 7.3) | 6.8 (5.9, |
| | | | | 7.7) |
| min-max | 4-10 | 3-10 | 4-9 | 3-10 |
| 95% CI | [6.5, 6.9] | [6.6, 7.3] | [6.1, 6.8] | [6.5, 6.9] |
| postherpetic | Qutenza ® | Qutenza ® | Qutenza ® | Qutenza ® |
| neuralgia | <84 days* | 84-109 days* | >110 days* | total |
| baseline | n = 46 | n = 20 | n = 19 | n = 85 |
| n | 46 | 20 | 19 | 85 |
| missing | 0 | 0 | 0 | 0 |
| mean (SD) | 6.7 (1.31) | 7.0 (1.62) | 6.1 (1.53) | 6.6 (1.45) |
| median (Q1, Q3) | 6.9 (6.0, 7.7) | 6.9 (5.6, 8.3) | 5.5 (4.8, 7.6) | 6.8 (5.5, |
| | | | | 7.7) |
| min-max | 4-10 | 5-10 | 4-9 | 4-10 |
| 95% CI | [6.3, 7.1] | [6.2, 7.8] | [5.4, 6.8] | [6.3, 6.9] |
| HIV associated | Qutenza ® | Qutenza ® | Qutenza ® | Qutenza ® |
| neuralgia | <84 days* | 84-109 days* | >110 days* | total |
| baseline | n = 14 | n = 19 | n = 18 | n = 51 |
| n | 14 | 19 | 18 | 51 |
| missing | 0 | 0 | 0 | 0 |
| mean (SD) | 6.8 (1.39) | 6.5 (1.29) | 6.5 (1.10) | 6.6 (1.24) |
| median (Q1, Q3) | 6.9 (6.0, 7.8) | 6.4 (6.2, 7.2) | 6.6 (6.0, 7.2) | 6.7 (6.0, |
| | | | | 7.2) |
| min-max | 4-9 | 3-9 | 4-8 | 3-9 |
| 95% CI | [6.0, 7.6] | [5.9, 7.2] | [5.9, 7.0] | [6.2, 6.9] |
| neuropathic | Qutenza ® | Qutenza ® | Qutenza ® | Qutenza ® |
| injury | <84 days* | 84-109 days* | >110 days* | total |
| baseline | n = 39 | n = 24 | n = 13 | n = 76 |
| n | 39 | 23 | 13 | 75 |
| missing | 0 | 1 | 0 | 1 |
| mean (SD) | 6.7 (1.35) | 7.2 (1.15) | 6.8 (1.20) | 6.9 (1.27) |
| median (Q1, Q3) | 6.8 (5.8, 7.9) | 7.0 (6.4, 7.9) | 6.4 (6.0, 8.0) | 6.9 (6.0, |
| | | | | 7.9) |
| min-max | 4-9 | 5-10 | 5-9 | 4-10 |
| 95% CI | [6.3, 7.2] | [6.7, 7.7] | [6.1, 7.6] | [6.6, 7.2] |
| other peripheral | Qutenza ® | Qutenza ® | Qutenza ® | Qutenza ® |
| neuropathic pain | <84 days* | 84-109 days* | >110 days* | total |
| baseline | n = 12 | n = 4 | n = 2 | n = 18 |
| n | 12 | 4 | 2 | 18 |
| missing | 0 | 0 | 0 | 0 |
| mean (SD) | 6.5 (1.04) | NA (NA) | NA (NA) | 6.6 (1.15) |
| median (Q1, Q3) | 6.6 (6.0, 7.0) | NA | NA | 6.7 (6.0, |
| | | | | 7.0) |
| min-max | 4-8 | NA | NA | 4-9 |
| 95% CI | [5.8, 7.2] | [NA, NA] | [NA, NA] | [6.1, 7.2] |

B) Subjects response after the first and the second application−30% decrease in NRS pain rating score compared to baseline:

| | Qutenza ® | Qutenza ® | Qutenza ® | |
|---|---|---|---|---|
| | <84 | 84-109 | >110 | Qutenza ® |
| overall | days* | days* | days* | total |
| number (percent) | n = 111 | n = 67 | n = 52 | n = 230 |
| number of subjects responding 12 weeks after | 24 (21.6) | 18 (26.9) | 22 (42.3) | 64 (27.8) |
| first or before second application | | | | |
| number of subjects responding 12 weeks after | 34 (30.6) | 24 (35.8) | 15 (28.8) | 73 (31.7) |
| second or before third application | | | | |
| relative change of response rate | +41.7 | +33.3 | −31.8 | +14.1 |
| | Qutenza ® | Qutenza ® | Qutenza ® | |
| | <84 | 84-109 | >110 | Qutenza ® |
| postherpetic neuralgia | days* | days* | days* | total |
| number (percent) | n = 46 | n = 20 | n = 19 | n = 85 |
| number of subjects responding 12 weeks after | 7 (15.2) | 5 (25.0) | 9 (47.4) | 21 (24.7) |
| first or before second application | | | | |
| number of subjects responding 12 weeks after | 13 (28.3) | 8 (40.0) | 5 (26.3) | 26 (30.6) |
| second or before third application | | | | |
| relative change of response rate | +85.7 | +60.0 | −44.4 | +23.8 |
| | Qutenza ® | Qutenza ® | Qutenza ® | |
| | <84 | 84-109 | >110 | Qutenza ® |
| HIV associated neuralgia | days* | days* | days* | total |
| number (percent) | n = 14 | n = 19 | n = 18 | n = 51 |
| number of subjects responding 12 weeks after | 5 (35.7) | 5 (26.3) | 6 (33.3) | 16 (31.4) |
| first or before second application | | | | |
| number of subjects responding 12 weeks after | 6 (42.9) | 6 (31.6) | 6 (33.3) | 18 (35.3) |
| second or before third application | | | | |
| relative change of response rate | +20.0 | +20.0 | 0.0 | +12.5 |
| | Qutenza ® | Qutenza ® | Qutenza ® | |
| | <84 | 84-109 | >110 | Qutenza ® |
| neuropathic injury | days* | days* | days* | total |
| number (percent) | n = 39 | n = 24 | n = 13 | n = 76 |
| number of subjects responding 12 weeks after | 11 (28.2) | 6 (25.0) | 7 (53.8) | 24 (31.6) |
| first or before second application | | | | |
| number of subjects responding 12 weeks after | 13 (33.3) | 8 (33.3) | 4 (30.8) | 25 (32.9) |
| second or before third application | | | | |
| relative change of response rate | +18.2 | +33.3 | −42.9 | +4.2 |
| | Qutenza ® | Qutenza ® | Qutenza ® | |
| | <84 | 84-109 | >110 | Qutenza ® |
| other peripheral neuropathic pain | days* | days* | days* | total |
| number (percent) | n = 12 | n = 4 | n = 2 | n = 18 |
| number of subjects responding 12 weeks after | 1 (8.3) | 2 (50.0) | 0 | 3 (16.7) |
| first or before second application | | | | |
| number of subjects responding 12 weeks after | 2 (16.7) | 2 (50.0) | 0 | 4 (22.2) |
| second or before third application | | | | |
| relative change of response rate | +100.0 | 0 | 0 | +33.3 |

C) Subjects response after the first and the second application−2 point decrease in NRS pain rating score compared to baseline:

| | Qutenza ® | Qutenza ® | Qutenza ® | |
|---|---|---|---|---|
| | <84 | 84-109 | >110 | Qutenza ® |
| overall | days* | days* | days* | total |
| number (percent) | n = 111 | n = 67 | n = 52 | n = 230 |
| number of subjects responding 12 weeks after | 27 (24.3) | 17 (25.4) | 20 (38.5) | 64 (27.8) |
| first or before second application | | | | |
| number of subjects responding 12 weeks after | 37 (33.3) | 26 (38.8) | 13 (25.0) | 76 (33.0) |
| second or before third application | | | | |
| relative change of response rate | +37.0 | +52.9 | −35.0 | +18.8 |
| | Qutenza ® | Qutenza ® | Qutenza ® | |
| | <84 | 84-109 | >110 | Qutenza ® |
| postherpetic neuralgia | days* | days* | days* | total |
| number (percent) | n = 46 | n = 20 | n = 19 | n = 85 |
| number of subjects responding 12 weeks after | 9 (19.6) | 4 (20.0) | 8 (42.1) | 21 (24.7) |
| first or before second application | | | | |
| number of subjects responding 12 weeks after | 15 (32.6) | 8 (40.0) | 4 (21.1) | 27 (31.8) |
| second or before third application | | | | |
| relative change of response rate | +66.7 | +100.0 | −50.0 | +28.6 |
| | Qutenza ® | Qutenza ® | Qutenza ® | |
| | <84 | 84-109 | >110 | Qutenza ® |
| HIV associated neuralgia | days* | days* | days* | total |
| number (percent) | n = 14 | n = 19 | n = 18 | n = 51 |
| number of subjects responding 12 weeks after | 7 (50.0) | 5 (26.3) | 7 (38.9) | 19 (37.3) |
| first or before second application | | | | |
| number of subjects responding 12 weeks after | 6 (42.9) | 6 (31.6) | 6 (33.3) | 18 (35.3) |
| second or before third application | | | | |
| relative change of response rate | −14.3 | +20.0 | −14.3 | −5.3 |
| | Qutenza ® | Qutenza ® | Qutenza ® | |
| | <84 | 84-109 | >110 | Qutenza ® |
| neuropathic injury | days* | days* | days* | total |
| number (percent) | n = 39 | n = 24 | n = 13 | n = 76 |
| number of subjects responding 12 weeks after | 9 (23.1) | 6 (25.0) | 5 (38.5) | 20 (26.3) |
| first or before second application | | | | |
| number of subjects responding 12 weeks after | 14 (35.9) | 10 (41.7) | 3 (23.1) | 27 (35.5) |
| second or before third application | | | | |
| relative change of response rate | +55.6 | +66.7 | −40.0 | +35.0 |
| | Qutenza ® | Qutenza ® | Qutenza ® | |
| | <84 | 84-109 | >110 | Qutenza ® |
| other peripheral neuropathic pain | days* | days* | days* | total |
| number (percent) | n = 12 | n = 4 | n = 2 | n = 18 |
| number of subjects responding 12 weeks after | 2 (16.7) | 2 (50.0) | 0 | 4 (22.2) |
| first or before second application | | | | |
| number of subjects responding 12 weeks after | 2 (16.7) | 2 (50.0) | 0 | 4 (22.2) |
| second or before third application | | | | |
| relative change of response rate | 0 | 0 | 0 | 0 |

D) Percent change from baseline in NRS pain rating score after the first and the second application:

| | | | | |
|---|---|---|---|---|
| | Qutenza ® | Qutenza ® | Qutenza ® | Qutenza ® |
| overall | <84 days* | 84-109 days* | >110 days* | total |
| baseline | n = 111 | n = 67 | n = 52 | n = 230 |

percent change from baseline 12 weeks after first or before second application

| | | | | |
|---|---|---|---|---|
| n | 110 | 62 | 47 | 219 |
| missing | 1 | 5 | 5 | 11 |
| mean (SD) | −13.6 (21.75) | −19.2 (19.62) | −28.0 (28.48) | −18.3 (23.40) |
| median (Q1, Q3) | −12.8 (−25.0, 0.0) | −14.7 (−35.5, −7.4) | −28.0 (−47.5, −4.5) | −15.2 (−33.3, 0.0) |
| min-max | −75-36 | −73-17 | −85-33 | −85-36 |
| 95% CI | [−17.7, −9.5] | [−24.2, −14.2] | [−36.4, −19.7] | [−21.4, −15.2] |

percent change from baseline 12 weeks after second or before third application

| n | 91 | 53 | 40 | 184 |
|---|---|---|---|---|
| missing | 20 | 12 | 12 | 46 |
| mean (SD) | −22.8 (26.80) | −25.4 (25.25) | −22.2 (31.34) | −23.4 (27.30) |
| median (Q1, Q3) | −20.8 (−42.9, −1.8) | −28.6 (−41.7, −5.1) | −20.0 (−40.6, 2.6) | −21.3 (−42.7, −1.9) |
| min-max | −100-50 | −86-29 | −100-33 | −100-50 |
| 95% CI | [−28.4, −17.2] | [−32.3, −18.4] | [−32.1, −12.1] | [−27.4, −19.4] |
| postherpetic | Qutenza ® | Qutenza ® | Qutenza ® | Qutenza ® |
| neuralgia | <84 days* | 84-109 days* | >110 days* | total |
| baseline | n = 46 | n = 20 | n = 19 | n = 85 |

percent change from baseline 12 weeks after first or before second application

| | | | | |
|---|---|---|---|---|
| n | 46 | 17 | 18 | 81 |
| missing | 0 | 3 | 1 | 4 |
| mean (SD) | −11.3 (18.93) | −17.6(18.04) | −26.7 (26.55) | −16.1 (21.34) |
| median (Ql, Q3) | −13.1 (−23.1, 0.0) | −10.0 (−33.3, −7.7) | −29.2 (−47.5, −2.3) | −14.3 (−30.6, 0.0) |
| min-max | −56-36 | −54-5 | −63-33 | −63-36 |
| 95% CI | [−17.0, −5.7] | [−26.8, −8.3] | [−39.9, −13.5] | [−20.8, −11.3] |

percent change from baseline 12 weeks after second or before third application

| n | 39 | 16 | 14 | 69 |
|---|---|---|---|---|
| missing | 7 | 4 | 5 | 16 |
| mean (SD) | −22.3 (21.12) | −26.3 (21.34) | −23.0(28.07) | −23.4 (22.43) |
| median (Ql, Q3) | −17.6 (−37.5, −6.7) | −26.1 (−40.5, −6.9) | −22.5 (−37.5, 0.0) | −20.5 (−37.5, −6.7) |
| min-max | −75-8 | −71 -5 | −76-33 | −76-33 |
| 95% CI | [−29.1, −15.4] | [−37.6, −14.9] | [−39.2, −6.8] | [−28.7, −18.0] |
| HIV associated | Qutenza ® | Qutenza ® | Qutenza ® | Qutenza ® |
| neuralgia | <84 days* | 84-109 days* | >110 days* | total |
| baseline | n = 14 | n = 19 | n = 18 | n = 51 |

percent change from baseline 12 weeks after first or before second application

| | | | | |
|---|---|---|---|---|
| n | 14 | 18 | 14 | 46 |
| missing | 0 | 1 | 4 | 5 |
| mean (SD) | −15.8 (30.08) | −17.6(14.83) | −29.9 (28.38) | −20.8 (24.79) |
| median (Ql, Q3) | −20.8 (−47.1, 14.3) | −14.3 (−35.5, −7.4) | −28.7 (−44.2, −4.5) | −21.1 (−37.3, −2.8) |
| min-max | −56-25 | −42-3 | −71-18 | −71 -25 |
| 95% CI | [−33.1, 1.6] | [−25.0, −10.2] | [−46.2, −13.5] | [−28.1, −13.4] |

percent change from baseline 12 weeks after second or before third application

| n | 9 | 14 | 13 | 36 |
|---|---|---|---|---|
| missing | 5 | 5 | 5 | 15 |
| mean (SD) | −34.2 (40.48) | −24.4 (25.94) | −28.0 (34.92) | −28.2 (32.52) |
| median (Ql, Q3) | −43.9 (−50.0,-22.6) | −25.1 (−51.6, 0.0) | −23.8 (−56.3, 0.0) | −31.6 (−51.6, 0.0) |
| min-max | −100-50 | −67-17 | −100-18 | −100-50 |
| 95% CI | [-65.3, −3.1] | [-39.4, −9.4] | [-49.1, −6.9] | [-39.2, −17.1] |
| neuropathic | Qutenza ® | Qutenza ® | Qutenza ® | Qutenza ® |
| injury | <84 days* | 84-109 days* | >110 days* | total |
| baseline | n = 39 | n = 24 | n = 13 | n = 76 |

percent change from baseline 12 weeks after first or before second application

| | | | | |
|---|---|---|---|---|
| n | 38 | 23 | 13 | 74 |
| missing | 1 | 1 | 0 | 2 |
| mean (SD) | −17.4 (22.99) | −20.6 (22.78) | −32.3 (31.78) | −21.0 (24.89) |
| median (Ql, Q3) | −13.6 (−30.8, 0.0) | −17.9 (−35.5, −7.9) | −30.4 (−63.0, −11.1) | −15.7 (−32.3, −4.0) |
| min-max | −75-29 | −73-17 | −85-17 | −85-29 |
| 95% CI | [−25.0, −9.9] | [−30.4, −10.7] | [−51.5, −13.1] | [−26.8, −15.2] |

percent change from baseline 12 weeks after second or before third application

| n | 33 | 20 | 11 | 64 |
|---|---|---|---|---|
| missing | 6 | 4 | 2 | 12 |
| mean (SD) | −24.1 (29.17) | −23.7(28.42) | −17.7 (34.49) | −22.9 (29.50) |
| median (Ql, Q3) | −22.2 (−48.1, 0.0) | −25.4 (−41.2, −5.9) | −8.6 (−33.3, 12.5) | −20.3 (−42.3, 0.7) |
| min-max | −82-26 | −86-29 | −81-17 | −86-29 |
| 95% CI | [−34.4, −13.7] | [−37.0, −10.4] | [−40.9, 5.5] | [−30.2, −15.5] |
| other peripheral | Qutenza ® | Qutenza ® | Qutenza ® | Qutenza ® |
| neuropathic pain | <84 days* | 84-109 days* | >110 days* | total |
| baseline | n = 12 | n = 4 | n = 2 | n = 18 |

percent change from baseline 12 weeks after first or before second application

| | | | | |
|---|---|---|---|---|
| n | 12 | 4 | 2 | 18 |
| missing | 0 | 0 | 0 | 0 |
| mean (SD) | −7.6 (16.10) | NA (NA) | NA (NA) | −10.7 (21.33) |
| median (Ql, Q3) | −2.2 (−19.4, 0.0) | NA | NA | −2.7 (−22.2, 0.0) |
| min-max | −34-25 | NA | NA | −63-25 |
| 95% CI | [−17.8,2.6] | [NA, NA] | [NA, NA] | [−21.3, −0.1] |

percent change from baseline 12 weeks after second or before third application

| | | | | |
|---|---|---|---|---|
| n | 10 | 3 | 2 | 15 |
| missing | 2 | 1 | 0 | 3 |
| mean (SD) | −10.6 (22.73) | NA (NA) | NA (NA) | −14.5 (24.43) |
| median (Ql, Q3) | −13.6 (−25.0, 0.0) | NA | NA | −12.5 (−34.0, 0.0) |
| min-max | −43-33 | NA | NA | −63-33 |
| 95% CI | [−26.9, 5.7] | [NA, NA] | [NA, NA] | [−28.0, −0.9] |

E) Change from baseline in NRS pain rating score after the first and the second application:

| | | | | |
|---|---|---|---|---|
| | Qutenza ® | Qutenza ® | Qutenza ® | Qutenza ® |
| overall | <84 days* | 84-109 days* | >110 days* | total |
| baseline | n = 111 | n = 67 | n = 52 | n = 230 |

percent change from baseline 12 weeks after first or before second application

| | | | | |
|---|---|---|---|---|
| n | 110 | 62 | 47 | 219 |
| missing | 1 | 5 | 5 | 11 |
| mean (SD) | −0.9 (1.43) | −1.3 (1.34) | −1.7 (1.80) | −1.2 (1.52) |
| median (Ql, Q3) | −0.9 (−1.9, 0.0) | −1.1 (−2.2, −0.4) | −1.6 (−3.0, −0.3) | −1.0 (−2.1, 0.0) |
| min-max | −6-2 | −5-1 | −6-2 | −6-2 |
| 95% CI | [−1.2, −0.7] | [−1.6, −1.0] | [−2.3, −1.2] | [−1.4, −1.0] |

percent change from baseline 12 weeks after second or before third application

| n | 91 | 53 | 40 | 184 |
|---|---|---|---|---|
| missing | 20 | 14 | 12 | 46 |
| mean (SD) | −1.5 (1.82) | −1.8 (1.82) | −1.3 (1.95) | −1.6(1.84) |
| median (Ql, Q3) | −1.3 (−2.8, −0.1) | −1.8 (−2.9, −0.4) | −1.0 (−2.2, 0.2) | −1.3 (−2.8, −0.1) |
| min-max | −9-2 | −7-2 | −7-2 | −9-2 |
| 95% CI | [−1.9, −1.2] | [−2.3, −1.3] | [−1.9, −0.7] | [−1.8, −1.3] |
| postherpetic | Qutenza ® | Qutenza ® | Qutenza ® | Qutenza ® |
| neuralgia | <84 days* | 84-109 days* | >110 days* | total |
| baseline | n = 46 | n = 20 | n = 19 | n = 85 |

percent change from baseline 12 weeks after first or before second application

| | | | | |
|---|---|---|---|---|
| n | 46 | 17 | 18 | 81 |
| missing | 0 | 3 | 1 | 4 |
| mean (SD) | −0.8 (1.20) | −1.2 (1.21) | −1.5 (1.42) | −1.0 (1.28) |
| median (Ql, Q3) | −0.9 (−1.5, 0.0) | −0.8 (−1.7, −0.5) | −1.4 (−2.8, −0.2) | −1.0 (−2.0, 0.0) |
| min-max | −4-2 | −4-0 | −4-2 | −4-2 |
| 95% CI | [−1.1, −0.4] | [−1.8, −0.6] | [−2.2, −0.8] | [−1.3, −0.7] |

percent change from baseline 12 weeks after second or before third application

| n | 39 | 16 | 14 | 69 |
|---|---|---|---|---|
| missing | 7 | 4 | 5 | 16 |
| mean (SD) | −1.5 (1.35) | −1.9 (1.51) | −1.2 (1.34) | −1.5 (1.39) |
| median (Ql, Q3) | −1.3 (−2.3, −0.4) | −2.1 (−2.9, −0.5) | −1.2 (−2.0, 0.0) | −1.3 (−2.5, −0.4) |
| min-max | −5-1 | −5-0 | −3-2 | −5-2 |
| 95% CI | [−1.9, −1.0] | [−2.7, −1.1] | [−1.9, −0.4] | [−1.8, −1.2] |
| HIV associated | Qutenza ® | Qutenza ® | Qutenza ® | Qutenza ® |
| neuralgia | <84 days* | 84-109 days* | >110 days* | total |
| baseline | n = 14 | n = 19 | n = 18 | n = 51 |

percent change from baseline 12 weeks after first or before second application

| | | | | |
|---|---|---|---|---|
| n | 14 | 18 | 14 | 46 |
| missing | 0 | 1 | 4 | 5 |
| mean (SD) | −1.1 (1.92) | −1.1 (0.87) | −1.9 (1.90) | −1.4 (1.59) |
| median (Ql, Q3) | −1.6 (−2.7, 1.0) | −1.1 (−2.0, −0.4) | −2.0 (−3.2, −0.3) | −1.3 (−2.3, −0.2) |
| min-max | −4-2 | −2-0 | −5-1 | −5-2 |
| 95% CI | [−2.2, −0.0] | [−1.5, −0.7] | [−3.0, −0.8] | [−1.8, −0.9] |

percent change from baseline 12 weeks after second or before third application

| n | 9 | 14 | 13 | 36 |
|---|---|---|---|---|
| missing | 5 | 5 | 5 | 15 |
| mean (SD) | −2.6 (2.91) | −1.7 (1.85) | −1.8 (2.35) | −2.0 (2.29) |
| median (Ql, Q3) | −2.7 (−3.1, −1.8) | −1.6 (−3.2, 0.0) | −1.3 (−3.9, 0.0) | −1.9 (−3.2, 0.0) |
| min-max | −9-2 | −6-1 | −7-1 | −9-2 |
| 95% CI | [−4.9, −0.4] | [−2.7, −0.6] | [−3.2, −0.4] | [−2.7, −1.2] |
| neuropathic | Qutenza ® | Qutenza ® | Qutenza ® | Qutenza ® |
| injury | <84 days* | 84-109 days* | >110 days* | total |
| baseline | n = 39 | n = 24 | n = 13 | n = 76 |

percent change from baseline 12 weeks after first or before second application

| | | | | |
|---|---|---|---|---|
| n | 38 | 23 | 13 | 74 |
| missing | 1 | 1 | 0 | 2 |
| mean (SD) | −1.2(1.59) | −1.5 (1.69) | −2.1 (2.13) | −1.4(1.74) |
| median (Ql, Q3) | −0.9 (−1.9, 0.0) | −1.3 (−2.8, −0.4) | −1.8 (−3.4, −1.0) | −1.0 (−2.1, −0.3) |
| min-max | −6-2 | −5-1 | −6-1 | −6-2 |
| 95% CI | [−1.7, −0.6] | [−2.2, −0.8] | [−3.4, −0.9] | [−1.8, −1.0] |

percent change from baseline 12 weeks after second or before third application

| n | 33 | 20 | 11 | 64 |
|---|---|---|---|---|
| missing | 6 | 4 | 2 | 12 |
| mean (SD) | −1.6 (1.96) | −1.8 (2.13) | −1.2 (2.30) | −1.6 (2.05) |
| median (Ql, Q3) | −1.3 (−3.0, 0.0) | −1.7 (−2.8, −0.5) | −0.8 (−2.0, 1.0) | −1.3 (−2.9, 0.0) |
| min-max | −6-1 | −7-2 | −6-1 | −7-2 |
| 95% CI | [−2.2, −0.9] | [−2.8, −0.8] | [−2.7, 0.4] | [−2.1, −1.0] |
| other peripheral | Qutenza ® | Qutenza ® | Qutenza ® | Qutenza ® |
| neuropathic pain | <84 days* | 84-109 days* | >110 days* | total |
| baseline | n = 12 | n = 4 | n = 2 | n = 18 |

percent change from baseline 12 weeks after first or before second application

| | | | | |
|---|---|---|---|---|
| n | 12 | 4 | 2 | 18 |
| missing | 0 | 0 | 0 | 0 |
| mean (SD) | −0.6 (1.02) | NA (NA) | NA (NA) | −0.7 (1.26) |
| median (Ql, Q3) | −0.2 (−1.2, 0.0) | NA | NA | −0.2 (−1.6, 0.0) |
| min-max | −3-1 | NA | NA | −3-1 |
| 95% CI | [−1.2,0.1] | [NA, NA] | [NA, NA] | [−1.4, −0.1] |

percent change from baseline 12 weeks after second or before third application

| | | | | |
|---|---|---|---|---|
| n | 10 | 3 | 2 | 15 |
| missing | 2 | 1 | 0 | 3 |
| mean (SD) | −0.7 (1.49) | NA (NA) | NA (NA) | −0.9 (1.51) |
| median (Ql, Q3) | −0.9 (−1.4, 0.0) | NA | NA | −0.9 (−2.6, 0.0) |
| min-max | −3-2 | NA | NA | −3-2 |
| 95% CI | [−1.8, 0.4] | [NA, NA] | [NA, NA] | [−1.8, −0.1] |

### Example 2

All patients were diagnosed with painful diabetic peripheral neuropathy. Repeat treatment with Qutenza® was allowed with intervals between treatments of at least 8 weeks.

At month 3, 48.6% of patients having received an initial treatment with high-concentration capsaicin patch (n=313) did not meet the responder criteria (n=152). Of these 152 patients, 127 (i.e. 83.5%) were still in the trial at 12 months, whereas from the total population, 250 patients (79.9%) were still in the trial. Of the initial non-responders receiving repeat applications, 80.2% had received at least 4 applications at month 12. At month 12, 45.7% of the initial non-responders who were still in the trial (n=127) had become responders to treatment, whereas of the total population 58% could be classified as a responder. The 58% refers to the total population at month 12, taking into account that not all patients remained in the trial for the full 12 months and some of them may have lost response over time.

An additional investigation into optimal repeated treatment interval showed that the percentage of subjects, responding after the first application (defined as a 30% decrease from baseline on the numeric pain rating scale (NRS)) was lower (21.6%) in those with the shorter treatment interval (<84 days) compared to the other subgroups with treatment intervals of ≥84 −<110 days or ≥110 days) with responder rates of 26.9% and 42.3% respectively, illustrating that the group with the smallest treatment interval included a higher number of non-responders. It is logical that a patient with a smaller treatment benefit would be retreated earlier (i.e. with shorter interval between treatments) in clinical practice as delaying treatment for such patients would mean that they would be in pain for a longer period of time. However, after the second application, the responder rates increased for the two groups with the lower treatment intervals (<84 days and ≥84−<110 days) to 30.6% and 35.8%, respectively.

In other words the responder rates increased with 41.7% in those with a <84 days treatment interval compared to 33.1% in those with the ≥84−<110 days interval. This outcome also supports that responder rates can increase with repeated applications.

As demonstrated by above Examples 1 and 2, repeat application also to initial non-responders provides additional benefit to patients. Across trials, more than one third of initial non-responders converted into responders upon repeated treatment with high-concentration capsaicin patch. Although at month 12, the responder rates in patients who did not initially respond to high-concentration capsaicin patch were approximately 10% lower than in the overall population, the substantial increase in responder rates to >30% in those who initially did not respond, justifies a repeat treatment.

1. A method for treating a neuropathic condition in a patient who upon previous topical administration of capsaicin and/or capsaicinoid did not perceive pain relief by at least 30% versus baseline, said method comprising topically administering to said patient one or more high-concentration capsaicin and/or capsaicinoid topical dose units comprising capsaicin and/or capsaicinoid at a concentration of at least 2.5 wt.-%, relative to a total weight of each dose unit.

2. The method according to claim 1, wherein the concentration of the capsaicin and/or capsaicinoid is at least 5 wt.-%, relative to the total weight of each dose unit.

3. The method according to claim 1, wherein the concentration of the capsaicin and/or capsaicinoid is at least 8 wt.-%, relative to the total weight of each dose unit.

4. The method according to claim 1, wherein the one or more high-concentration capsaicin and/or capsaicinoid topical dose units are applied to the skin of the patient, whereby the patient perceives pain relief by at least 30% versus baseline.

5. The method according to claim 1, wherein the one or more high-concentration capsaicin and/or capsaicinoid topical dose units are applied to the skin of the patient, whereby the patient perceives pain relief by at least 30% versus baseline for at least 8 weeks.

6. The method according to claim 1, wherein with regard to the previous topical administration of capsaicin and/or capsaicinoid the patient is selected from complete non-responders and insufficient responders.

7. The method according to claim 1, wherein the one or more high-concentration capsaicin and/or capsaicinoid topical dose units are applied to the skin of the patient for an application period of 15 to 90 minutes and subsequently removed.

8. The method according to claim 7, wherein the application period is 30 to 60 minutes.

9. The method according to claim 1, wherein the one or more high-concentration capsaicin and/or capsaicinoid topical dose units are applied to the skin of the patient 2 to 4 months after previous topical administration of capsaicin and/or capsaicinoid.

10. The method according to claim 9, wherein the one or more high-concentration capsaicin and/or capsaicinoid topical dose units are applied to the skin of the patient less than 90 days after previous topical administration of capsaicin and/or capsaicinoid.

11. The method according to claim 9, wherein the one or more high-concentration capsaicin and/or capsaicinoid topical dose units are applied to the skin of the patient about 3 months after previous topical administration of capsaicin and/or capsaicinoid.

12. The method according to claim 1, wherein the patient did not perceive pain relief by at least 30% versus baseline upon repeated previous topical administrations of capsaicin and/or capsaicinoid.

13. The method according to claim 12, wherein the one or more high-concentration capsaicin and/or capsaicinoid topical dose units are applied to the skin of the patient 2 to 4 months after the last topical administration of said repeated previous topical administrations.

14. The method according to claim 13, wherein the one or more high-concentration capsaicin and/or capsaicinoid topical dose units are applied to the skin of the patient less than 90 days after the last topical administration of said repeated previous topical administrations.

15. The method according to claim 13, wherein the one or more high-concentration capsaicin and/or capsaicinoid topical dose units are applied to the skin of the patient about 3 months after the last topical administration of said repeated previous topical administrations.

16. The method according to claim 1, wherein previous topical administration of capsaicin and/or capsaicinoid was achieved or wherein repeated previous topical administrations were achieved by applying one or more high-concentration capsaicin and/or capsaicinoid topical dose units to the skin of the patient.

17. The method according to claim 16, wherein said one or more high-concentration capsaicin and/or capsaicinoid topical dose units are of a same kind as were used in said previous topical administration.

18. The method according to claim 1,
wherein previous topical administration of capsaicin and/or capsaicinoid was achieved by applying one or more high-concentration capsaicin and/or capsaicinoid topical dose units comprising capsaicin and/or capsaicinoid to the skin of the patient for a first application period of 15 to 90 minutes thereby previously topically administering capsaicin and/or capsaicinoid during the first application period, and were subsequently removed, whereby the patient did not perceive pain relief by at least 30% versus baseline; and
wherein
(b-i) 2 to 4 months after the first application period, one or more high-concentration capsaicin and/or capsaicinoid topical dose units comprising capsaicin and/or capsaicinoid are applied to the skin of the patient, remain on the skin for a second application period of 15 to 90 minutes thereby topically administering capsaicin and/or capsaicinoid during the second application period, and are subsequently removed; and
(b-ii) optionally, 2 to 4 months after the second application period, one or more high-concentration capsaicin and/or capsaicinoid topical dose units comprising capsaicin and/or capsaicinoid are applied to the skin of the patient, remain on the skin for a third application period of 15 to 90 minutes thereby topically administering capsaicin and/or capsaicinoid during the third application period, and are subsequently removed; and
(b-iii) optionally, 2 to 4 months after the third application period, one or more high-concentration capsaicin and/or capsaicinoid topical dose units comprising capsaicin and/or capsaicinoid are applied to the skin of the patient, remain on the skin for a fourth application period of 15 to 90 minutes thereby topically administering capsaicin and/or capsaicinoid during the fourth application period, and are subsequently removed; and
(b-iv) optionally, 2 to 4 months after the fourth application period, one or more high-concentration capsaicin and/or capsaicinoid topical dose units comprising capsaicin and/or capsaicinoid are applied to the skin of the patient, remain on the skin for a fifth application period of 15 to 90 minutes thereby topically administering capsaicin and/or capsaicinoid during the fifth application period, and are subsequently removed.

19. The method according to claim 18, wherein
(b-i) less than 90 days after the first application period, one or more high-concentration capsaicin and/or capsaicinoid topical dose units comprising capsaicin and/or capsaicinoid are applied to the skin of the patient, remain on the skin for a second application period of 15 to 90 minutes thereby topically administering capsaicin and/or capsaicinoid during the second application period, and are subsequently removed; and
(b-ii) optionally, less than 90 days after the second application period, one or more high-concentration capsaicin and/or capsaicinoid topical dose units comprising capsaicin and/or capsaicinoid are applied to the skin of the patient, remain on the skin for a third application period of 15 to 90 minutes thereby topically administering capsaicin and/or capsaicinoid during the third application period, and are subsequently removed; and
(b-iii) optionally, less than 90 days after the third application period, one or more high-concentration capsaicin and/or capsaicinoid topical dose units comprising capsaicin and/or capsaicinoid are applied to the skin of the patient, remain on the skin for a fourth application period of 15 to 90 minutes thereby topically administering capsaicin and/or capsaicinoid during the fourth application period, and are subsequently removed; and
(b-iv) optionally, less than 90 days after the fourth application period, one or more high-concentration capsaicin and/or capsaicinoid topical dose units comprising capsaicin and/or capsaicinoid are applied to the skin of the patient, remain on the skin for a fifth application period of 15 to 90 minutes thereby topically administering capsaicin and/or capsaicinoid during the fifth application period, and are subsequently removed.

20. The method according to claim 18, wherein
(b-i) about 3 months after the first application period, one or more high-concentration capsaicin and/or capsaicinoid topical dose units comprising capsaicin and/or capsaicinoid are applied to the skin of the patient, remain on the skin for a second application period of 15 to 90 minutes thereby topically administering capsaicin and/or capsaicinoid during the second application period, and are subsequently removed; and
(b-ii) optionally, about 3 months after the second application period, one or more high-concentration capsaicin and/or capsaicinoid topical dose units comprising capsaicin and/or capsaicinoid are applied to the skin of the patient, remain on the skin for a third application period of 15 to 90 minutes thereby topically administering capsaicin and/or capsaicinoid during the third application period, and are subsequently removed; and
(b-iii) optionally, about 3 months after the third application period, one or more high-concentration capsaicin and/or capsaicinoid topical dose units comprising capsaicin and/or capsaicinoid are applied to the skin of the patient, remain on the skin for a fourth application period of 15 to 90 minutes thereby topically administering capsaicin and/or capsaicinoid during the fourth application period, and are subsequently removed; and
(b-iv) optionally, about 3 months after the fourth application period, one or more high-concentration capsaicin and/or capsaicinoid topical dose units comprising capsaicin and/or capsaicinoid are applied to the skin of the patient, remain on the skin for a fifth application period of 15 to 90 minutes thereby topically administering capsaicin and/or capsaicinoid during the fifth application period, and are subsequently removed.

21. The method according to claim 1, wherein each of said one or more high-concentration capsaicin and/or capsaicinoid topical dose units is a high-concentration capsaicin and/or capsaicinoid pharmaceutical patch comprising capsaicin and/or capsaicinoid at a concentration of at least 2.5 wt.-%, relative to the total weight of the patch without release liner.

22. The method according to claim 21, wherein the concentration of the capsaicin and/or capsaicinoid is at least 5 wt.-%, relative to the total weight of the patch without release liner.

23. The method according to claim 21, wherein the concentration of the capsaicin and/or capsaicinoid is at least 8 wt.-%, relative to the total weight of the patch without release liner.

24. The method according to claim 21, wherein each high-concentration capsaicin and/or capsaicinoid pharmaceutical patch comprises a backing layer, an adhesive layer, and a release liner; and wherein the content of capsaicin and/or capsaicinoid is at least 6.0 wt.-%, relative to the total weight of the pharmaceutical patch without release liner.

25. The method according to claim 24, wherein the content of capsaicin and/or capsaicinoid is about 8.0 wt.-%, relative to the total weight of the pharmaceutical patch without release liner.

26. The method according to claim 21, wherein each high-concentration capsaicin and/or capsaicinoid pharmaceutical patch comprises capsaicin and/or capsaicinoid at a content of at least 400 micrograms of capsaicin and/or capsaicinoid per cm² of patch.

27. The method according to claim 26, wherein the content is about 640 micrograms of capsaicin and/or capsaicinoid per cm² of patch.

28. The method according to claim 1, wherein each of said one or more high-concentration capsaicin and/or capsaicinoid topical dose units is a high-concentration capsaicin topical dose unit.

29. The method according to claim 1, which is performed for relief of neuropathic pain.

30. The method according to claim 29, wherein the neuropathic pain is peripheral neuropathic pain.

31. The method according to claim 29, wherein the neuropathic pain is selected from the group consisting of painful diabetic neuropathy, postherpetic neuralgia, chemotherapy-induced neuropathic pain, HIV-associated neuropathy, small-fiber neuropathy, chronic idiopathic axonal polyneuropathy, post-traumatic neuropathic pain, and post-surgical neuropathic pain.

32. The method according to claim 1, which is performed for relief of neuropathic pain selected from the group consisting of postherpetic neuralgia, chemotherapy-induced neuropathic pain, HIV-associated neuropathy, small-fiber neuropathy, chronic idiopathic axonal polyneuropathy, posttraumatic neuropathic pain, and post-surgical neuropathic pain.

33. The method according to claim 1, which is performed for regenerating and/or restoring sensory nerve fibers.
